(19) United States
(12) Patent Application Publication
(10) Pub. No.: US 2022/0134035 A1
(43) Pub. Date: May 5, 2022

(54) METHODS AND APPARATUS FOR OPERATING AN OXYGEN CONCENTRATOR

(71) Applicants: ResMed Asia Pte. Ltd., Singapore (SG); ResMed Pty Ltd, Bella Vista (AU)

(72) Inventors: Shayan Miaralipour, Sydney (AU); Rex Dael Navarro, Singapore (SG); Jason Yuqian Huang, Singapore (SG); Kean Wah Low, Singapore (SG)

(73) Assignees: ResMed Asia Pte. Ltd., Singapore (SG); ResMed Pty Ltd, Bella Vista (AU)

(21) Appl. No.: 17/470,381

(22) Filed: Sep. 9, 2021

Related U.S. Application Data
(60) Provisional application No. 63/109,092, filed on Nov. 3, 2020.

(51) Int. Cl. A61M 16/00; A61M 16/10; B01D 53/047

(52) U.S. Cl. CPC A61M 16/024; A61M 16/101; A61M 16/0003; B01D 53/047; A61M 2016/0027; B01D 2259/40009; A61M 2205/3584; B01D 2253/108; B01D 2256/12; B01D 2257/102; A61M 2205/502

(57) ABSTRACT

Oxygen concentrator methods and apparatus estimate sieve bed effective capacity. Estimation applies function(s) to a parameter of a measured pressure-time characteristic of the bed, characteristic of a phase of an adsorption cycle of the concentrator at a predetermined motor speed of its compression system. Estimation may involve operating the concentrator at a predetermined bed pressure and measuring a mass flow of gas entering or exiting the bed, and may use the measured mass flow and one or more functions. Estimation may involve a measured bed exhaust mass flow for a purge phase when bed pressure is regulated to maintain a predetermined target pressure using motor speed adjustment. The estimation may apply exhaust mass flow function(s) to the measured exhaust mass flow. Estimation of the effective capacity may involve applying motor speed function(s) to measured motor speed, such as an adjusted one for regulating canister pressure to achieve a target pressure.

### CROSS-REFERENCE TO RELATED APPLICATIONS

This application claims the benefit of the filing date of U.S. Provisional Patent Application No. 63/109,092 filed Nov. 3, 2020, the disclosure of which is hereby incorporated herein by reference.

### FIELD OF THE TECHNOLOGY

The present technology relates generally to methods and apparatus for treating respiratory disorders, such as those involving controlled pressure swing adsorption to generate oxygen enriched air. Such methodologies may be implemented in an oxygen concentrator. In some examples, the technology more specifically concerns such methods and apparatus for estimating the effective capacity of a selective adsorption system used by an oxygen concentrator for supplying oxygen enriched air to patients with respiratory disorders.

### DESCRIPTION OF THE RELATED ART

### Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Examples of respiratory disorders include respiratory failure, Obesity Hyperventilation Syndrome (OHS), Chronic Obstructive Pulmonary Disease (COPD), Neuromuscular Disease (NMD) and Chest wall disorders.

Respiratory failure is an umbrella term for respiratory disorders in which the lungs are unable to inspire sufficient oxygen or exhale sufficient CO₂ to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive. Rapidly progressive disorders are characterised by muscle impairment that worsens over months and results in death within a few years (e.g. Amyotrophic lateral sclerosis (ALS) and Duchenne muscular dystrophy (DMD) in teenagers). Variable or slowly progressive disorders are characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

### Respiratory Therapies

Various respiratory therapies, such as Non-invasive ventilation (NIV), Invasive ventilation (IV), and High Flow Therapy (HFT) have been used to treat one or more of the above respiratory disorders.

### Respiratory Pressure Therapies

Respiratory pressure therapy (RPT) is the application of a supply of air to an entrance to the airways at a controlled target pressure that is nominally positive with respect to atmosphere throughout the patient's breathing cycle (in contrast to negative pressure therapies such as the tank ventilator or cuirass).

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube. In some forms, the comfort and effectiveness of these therapies may be improved.

### Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched air. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient's peak inspiratory flow rate. HFT has been used to treat respiratory failure, COPD, and other respiratory disorders. One mechanism of action is that the high flow rate of air at the airway entrance improves ventilation efficiency by flushing, or washing out, expired CO₂ from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

### Respiratory Therapy Systems

These respiratory therapies may be provided by a respiratory therapy system or device. Such systems and devices may also be used to screen, diagnose, or monitor a condition without treating it.

A respiratory therapy system as described herein may comprise an oxygen source, an air circuit, and a patient interface.

### Oxygen Source

Experts in this field have recognized that exercise for respiratory failure patients provides long term benefits that slow the progression of the disease, improve quality of life and extend patient longevity. Most stationary forms of exercise like tread mills and stationary bicycles, however, are too strenuous for these patients. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds when mounted.

Oxygen concentrators have been in use for about 50 years to supply oxygen for respiratory therapy. Oxygen concentrators may implement cyclic processes such as vacuum swing adsorption (VSA), pressure swing adsorption (PSA), or vacuum pressure swing adsorption (VPSA). For example, oxygen concentrators may work based on depressurization (e.g., vacuum operation) and/or pressurization (e.g., compressor operation) in a swing adsorption process (e.g., Vacuum Swing Adsorption, Pressure Swing Adsorption or Vacuum Pressure Swing Adsorption, each of which are referred to herein as a “swing adsorption process”). Pressure swing adsorption may involve using one or more compressors to increase gas pressure inside one or more canisters that contains particles of a gas separation adsorbent. Such a canister when containing a mass of gas separation adsorbent such as a layer of gas separation adsorbent may be referred to as a sieve bed. As the pressure increases, certain molecules in the gas may become adsorbed onto the gas separation adsorbent. Removal of a portion of the gas in the canister under the pressurized conditions allows separation of the non-adsorbed molecules from the adsorbed molecules. The adsorbed molecules may then be desorbed by venting the canisters to ambient. Further details regarding oxygen concentrators may be found, for example, in U.S. Published Patent Application No. 2009-0065007, published Mar. 12, 2009, and entitled “Oxygen Concentrator Apparatus and Method”, which is incorporated herein by reference.

Ambient air usually includes approximately 78% nitrogen and 21% oxygen with the balance comprised of argon, carbon dioxide, water vapor and other trace gases. If a feed gas mixture such as air, for example, is fed under pressure through a canister containing a gas separation adsorbent that attracts nitrogen more strongly than it does oxygen, part or all of the nitrogen will be adsorbed by the adsorbent, and the gas coming out of the canister will be enriched in oxygen. When the adsorbent reaches the end of its capacity to adsorb nitrogen, the adsorbed nitrogen may be desorbed by venting. The canister is then ready for another cycle of producing oxygen enriched air. By alternating pressurization of the canisters in a two-canister system, one canister can be separating (or concentrating) oxygen (the “adsorption phase”) while the other canister is being vented (resulting in a near-continuous separation of oxygen from the air). This alternation results in a near-continuous separation of the oxygen from the nitrogen. In this manner, oxygen enriched air can be accumulated, such as in a storage container or other pressurizable vessel or conduit coupled to the canisters, for a variety of uses including providing supplemental oxygen to users.

Vacuum swing adsorption (VSA) provides an alternative gas separation technique. VSA typically draws the gas through the separation process of the canisters using a vacuum such as a compressor configured to create a vacuum within the canisters. Vacuum Pressure Swing Adsorption (VPSA) may be understood to be a hybrid system using a combined vacuum and pressurization technique. For example, a VPSA system may pressurize the canisters for the separation process and also apply a vacuum for depressurizing the canisters.

Traditional oxygen concentrators have been bulky and heavy making ordinary ambulatory activities with them difficult and impractical. Recently, companies that manufacture large stationary oxygen concentrators began developing portable oxygen concentrators (POCs). The advantage of POCs is that they can produce a theoretically endless supply of oxygen and provide mobility for patients (users). In order to make these devices small for mobility, the various systems necessary for the production of oxygen enriched air are condensed. POCs seek to utilize their produced oxygen as efficiently as possible, in order to minimise weight, size, and power consumption. In some implementations, this may be achieved by delivering the oxygen enriched air as series of pulses. This therapy mode is known as pulsed oxygen delivery (POD) or demand mode, in contrast with traditional continuous flow delivery more suited to stationary oxygen concentrators. POD mode may be implemented with a conserver, which is essentially an active valve with a sensor to determine when to release each bolus.

### Air Circuit

An air circuit is a conduit or a tube constructed and arranged to allow, in use, a flow of air to travel between two components of a respiratory therapy system such as the oxygen source and the patient interface. In some cases, there may be separate limbs of the air circuit for inhalation and exhalation. In other cases, a single limb air circuit is used for both inhalation and exhalation.

### Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

### Sieve Bed Degradation

The gas separation adsorbents used in POCs have a very high affinity for water. This affinity is so high that it overcomes nitrogen affinity, and thus when both water vapor and nitrogen are available in a feed gas stream (such as ambient air), the adsorbent will preferentially adsorb water vapor over nitrogen. Furthermore, when it is adsorbed, water does not desorb as easily as nitrogen. As a result, water molecules remain adsorbed even after regeneration and thus block the adsorption sites for nitrogen. Therefore, over time and use, water accumulates on the adsorbent, which becomes less and less efficient for nitrogen adsorption. The sieve bed thus degrades over time. Once the sieve bed is so degraded that it is unable to concentrate oxygen above a certain percentage threshold of purity, it is referred to as fully degraded and needs to be replaced. The remaining effective nitrogen-adsorbing capacity of a sieve bed is generally inverse to its state of degradation, in that a fresh sieve bed has an effective capacity of 100%, while a fully degraded sieve bed has an effective capacity of zero.

Previous attempts to use various performance parameters of a portable oxygen concentrator such as output oxygen purity to estimate the effective capacity of a sieve bed have relied on heuristics and as such, tend to be inaccurate, particularly in the later stages of use of a sieve bed.

It would be advantageous to be able to estimate the effective capacity of the sieve bed(s) of a portable oxygen concentrator more accurately. Users could then be kept informed of the effective capacity in order to plan for replacement of a fully degraded sieve bed or a sieve bed approaching full degradation.

### Summary of the Technology

Examples of the present technology may provide methods and apparatus for controlled operations of an oxygen concentrator, such as a portable oxygen concentrator. In particular, the technology provides methods and apparatus for a portable oxygen concentrator having an effective capacity measurement mode to estimate the effective capacity of a sieve bed. As the adsorbent in a sieve bed becomes degraded, it is less able to adsorb nitrogen from the feed gas stream. This difference manifests over time as changes in a number of measurable operational parameters of the portable oxygen concentrator. The disclosed methods and apparatus extract one or more of these indicative parameters during the effective capacity measurement mode and estimate the effective capacity of the sieve beds based on the extracted parameters.

Examples of the present technology also provide methods and apparatus for controlled operations of an oxygen concentrator, such as a portable oxygen concentrator. In particular, the technology provides methods and apparatus for a portable oxygen concentrator to estimate the effective capacity of a sieve bed during normal, pressure-regulated operation. As the adsorbent in a sieve bed becomes degraded, it is less able to adsorb nitrogen from the feed gas stream. This difference manifests over time as changes in a number of operational parameters of the portable oxygen concentrator that are measurable during normal operation. The disclosed methods and apparatus extract one or more of these indicative parameters during normal operation and estimate the effective capacity of the sieve beds based on the extracted parameters.

Some implementations of the present technology may include a method of estimating effective capacity of a sieve bed in an oxygen concentrator. The method may include accessing a parameter of a measured pressure-time characteristic of the sieve bed for a phase of a pressure swing adsorption cycle of the oxygen concentrator at a predetermined speed of a motor of a compression system of the oxygen concentrator. The method may include accessing one or more functions of the parameter of the measured pressure-time characteristic. The method may include estimating the effective capacity by applying the one or more functions to the parameter of the measured pressure-time characteristic.

In some implementations, the one or more functions use a fresh value of the parameter. The fresh value may be a value of the parameter obtained from a fresh sieve bed of a same type as the sieve bed at the predetermined speed of the motor. The one or more functions may use a fully degraded value of the parameter. The fully degraded value may be a value of the parameter obtained from a fully degraded sieve bed of a same type as the sieve bed at the predetermined speed of the motor. The one or more functions may comprise an interpolation using the fresh value of the parameter and the fully degraded value of the parameter. The parameter may be an initial rate of increase of the measured pressure-time characteristic. The parameter may be a rise time of the pressure-time characteristic. The phase may be a pressurisation phase of the pressure swing adsorption cycle.

The method may further include measuring the pressure-time characteristic of the sieve bed for the phase of the pressure swing adsorption cycle of the oxygen concentrator. The measuring may use a pressure in an accumulator of the oxygen concentrator. The measuring may use a power parameter of a control signal of the motor. The method may include repeating the accessing and the estimating to obtain a further estimate of effective capacity, and estimating a remaining usage time of the sieve bed from the estimate and the further estimate of effective capacity. The method may include displaying, on a display of the oxygen concentrator, an indicator of the estimated effective capacity. The method may include generating a message based on the estimated effective capacity.

Some implementations of the present technology may include an oxygen concentrator. The oxygen concentrator may include a sieve bed containing a gas separation adsorbent. The oxygen concentrator may include a compression system configured to feed a feed gas into the sieve bed. The oxygen concentrator may include a memory. The oxygen concentrator may include a controller. The controller may include one or more processors. The one or more processors may be configured by program instructions stored in the memory to execute an of the method of estimating effective capacity of the sieve bed as described herein.

Some implementations of the present technology may include an oxygen concentrator. The oxygen concentrator may include a sieve bed containing a gas separation adsorbent. The oxygen concentrator may include a compression system configured to feed a feed gas into the sieve bed. The oxygen concentrator may include a memory. The oxygen concentrator may include a controller. The controller may be configured to access a parameter of a measured pressure-time characteristic of the sieve bed for a phase of a pressure swing adsorption cycle of the oxygen concentrator at a predetermined speed of a motor of a compression system of the oxygen concentrator. The controller may be configured to access one or more functions of the parameter of the measured pressure-time characteristic. The controller may be configured to estimate effective capacity of the sieve bed by applying the one or more functions to the parameter of the measured pressure-time characteristic.

Some implementations of the present technology may include a connected oxygen therapy system. The system may include a portable oxygen concentrator that may include a sieve bed containing a gas separation adsorbent. The system may include an external computing device in communication with the portable oxygen concentrator. The system may include a memory. The system may include a processor configured by program instructions stored in the memory to estimate effective capacity of the sieve bed. The processor may be configured to access a parameter of a measured pressure-time characteristic of the sieve bed for a phase of a pressure swing adsorption cycle of the oxygen concentrator at a predetermined speed of a motor of a compression system of the oxygen concentrator. The processor may be configured to access one or more functions of the parameter of the measured pressure-time characteristic. The processor may be configured to estimate effective capacity of the sieve bed by applying the one or more functions to the parameter of the measured pressure-time characteristic.

In some implementations, the processor and the memory may be part of the portable oxygen concentrator. The processor may be further configured to transmit the effective capacity estimate to the external computing device. The processor and the memory may be part of the external computing device. The system may include a display. The processor may be further configured to display an indicator of the effective capacity that may be estimated on the display. The external computing device may be a portable computing device. The external computing device may be a server. The system may include a personal computing device in communication with the server. The personal computing device may be configured to interact with a portal system hosted by the server. The personal computing device may be configured to receive the effective capacity estimate from the portal system. The personal computing device may be configured to display the effective capacity estimate on a display of the personal computing device. The system may include a portable computing device in communication with the server. The portable computing device may be configured to receive the effective capacity estimate from the server. The portable computing device may be configured to display the effective capacity estimate on a display of the portable computing device.

Some implementations of the present technology may include apparatus. The apparatus may include means for accessing a parameter of a measured pressure-time characteristic of a sieve bed for a phase of a pressure swing adsorption cycle of an oxygen concentrator at a predetermined speed of a motor of a compression system of the oxygen concentrator. The apparatus may include means for accessing one or more functions of the parameter of the measured pressure-time characteristic. The apparatus may include means for estimating effective capacity of the sieve bed by applying the one or more functions to the parameter of the measured pressure-time characteristic.

Some implementations of the present technology may include a method of estimating effective capacity of a sieve bed in an oxygen concentrator. The method may include operating the oxygen concentrator to pressurise the sieve bed to a predetermined pressure. The apparatus may include accessing a measure comprising a mass flow of gas entering or exiting the pressurised sieve bed. The apparatus may include estimating the effective capacity using the measure of mass flow and one or more functions.

In some implementations, the method may comprise measuring the mass flow of gas wherein the measure comprises a mass flow of gas exiting the pressurised sieve bed, and the measuring includes controlling opening of a supply valve to permit gas to exit the pressurised sieve bed. The one or more functions may use a fresh value of the mass flow. The fresh value may be a value of the mass flow obtained from a fresh sieve bed of a same type as the sieve bed. The one or more functions may use a fully degraded value of the mass flow. The fully degraded value may be a value of the mass flow obtained from a fully degraded sieve bed of a same type as the sieve bed. The one or more functions may comprise an interpolation using the fresh value of the mass flow and the fully degraded value of the mass flow. The method may include repeating the pressurising, measuring, and estimating to obtain a further estimate of effective capacity. The method may include estimating a remaining usage time of the sieve bed from the estimate and the further estimate of effective capacity. The method may include displaying, on a display of the oxygen concentrator, an indicator of the estimated effective capacity. The method may include generating a message based on the estimated effective capacity.

Some implementations of the present technology may include an oxygen concentrator. The oxygen concentrator may include a sieve bed containing a gas separation adsorbent. The oxygen concentrator may include a compression system configured to feed a feed gas into the sieve bed. The oxygen concentrator may include a memory. The oxygen concentrator may include a controller. The controller may be configured to operate the compression system to pressurise the sieve bed to a predetermined pressure. The controller may be configured to access a measure comprising a mass flow of gas entering or exiting the pressurised sieve bed. The controller may be configured to estimate effective capacity using the measure of mass flow and one or more functions.

Some implementations of the present technology may include a connected oxygen therapy system. The system may include a portable oxygen concentrator configured to pressurize a sieve bed containing a gas separation adsorbent. The system may include an external computing device in communication with the portable oxygen concentrator. The system may include a memory. The system may include a processor configured by program instructions stored in the memory to estimate effective capacity of the sieve bed. The processor may be configured to access a measure may include a mass flow of gas entering or exiting the pressurised sieve bed. The measure may be measured during an operation of the oxygen concentrator that pressurises the sieve bed to a predetermined pressure. The processor may be configured to estimate effective capacity using the measure of mass flow and one or more functions.

In some implementations, the processor and the memory may be part of the portable oxygen concentrator. The processor may be further configured to transmit the effective capacity estimate to the external computing device. The processor and the memory may be part of the external computing device. The system may include a display. The processor may be further configured to display an indicator of the effective capacity that may be estimated on the display. The external computing device may be a portable computing device. The external computing device may be a server. The system may include a personal computing device in communication with the server. The personal computing device may be configured to interact with a portal system hosted by the server. The personal computing device may be configured to receive the effective capacity estimate from the portal system. The personal computing device may be configured to display the effective capacity estimate on a display of the personal computing device. The system may include a portable computing device in communication with the server. The portable computing device may be configured to receive the effective capacity estimate from the server. The personal computing device may be configured to display the effective capacity estimate on a display of the portable computing device.

Some implementations of the present technology may include apparatus. The apparatus may include means for pressurising a sieve bed to a predetermined pressure. The apparatus may include means for accessing a measure of a mass flow of gas entering or exiting the pressurised sieve bed. The apparatus may include means for estimating effective capacity using the measure of mass flow and one or more functions.

Some implementations of the present technology may include a method of estimating effective capacity of a sieve bed in an oxygen concentrator. The method may include accessing a measured exhaust mass flow of the sieve bed for a purge phase of a pressure swing adsorption cycle of the oxygen concentrator where the pressure in the sieve bed may be regulated to maintain a predetermined target pressure by adjusting the speed of a motor of a compression system of the oxygen concentrator. The method may include accessing one or more exhaust mass flow functions. The method may include estimating the effective capacity by applying the one or more exhaust mass flow functions to the measured exhaust mass flow.

The one or more exhaust mass flow functions use a fresh value of the exhaust mass flow, wherein the fresh value may be a value of the exhaust mass flow obtained from a fresh sieve bed of a same type as the sieve bed at the predetermined target pressure. The one or more exhaust mass flow functions use a fully degraded value of the exhaust mass flow. The fully degraded value may be a value of the exhaust mass flow obtained from a fully degraded sieve bed of a same type as the sieve bed at the predetermined target pressure. The one or more exhaust mass flow functions comprise an interpolation using the fresh value of the exhaust mass flow and the fully degraded value of the exhaust mass flow. The method may include correcting the exhaust mass flow for a purge mass flow from a further sieve bed in the oxygen concentrator over the purge phase. The method may include repeating the accessing and the estimating to obtain a further estimate of effective capacity. The method may include estimating a remaining usage time of the sieve bed from the estimate and the further estimate of effective capacity. The method may include displaying, on a display of the oxygen concentrator, an indicator of the estimated effective capacity. The method may include generating a message based on the estimated effective capacity.

Some implementations of the present technology may include an oxygen concentrator. The oxygen concentrator may include a sieve bed containing a gas separation adsorbent. The oxygen concentrator may include a compression system configured to feed a feed gas into the sieve bed. The oxygen concentrator may include a memory. The oxygen concentrator may include a controller. The controller may be configured to access a measured exhaust mass flow of the sieve bed for a purge phase of a pressure swing adsorption cycle of the oxygen concentrator where the pressure in the sieve bed may be regulated to maintain a predetermined target pressure by adjusting the speed of a motor of a compression system of the oxygen concentrator. The controller may be configured to access one or more exhaust mass flow functions. The controller may be configured to estimate effective capacity by applying the one or more exhaust mass flow functions to the measured exhaust mass flow.

Some implementations of the present technology may include a connected oxygen therapy system. The system may include a portable oxygen concentrator may include a sieve bed containing a gas separation adsorbent. The system may include an external computing device in communication with the portable oxygen concentrator. The system may include memory. The system may include a processor configured by program instructions stored in the memory to estimate effective capacity of the sieve bed. The processor configured to access a measured exhaust mass flow of the sieve bed for a purge phase of a pressure swing adsorption cycle of the oxygen concentrator, where the pressure in the sieve bed may be regulated to maintain a predetermined target pressure by adjusting the speed of a motor of a compression system of the oxygen concentrator. The processor configured to access one or more exhaust mass flow functions. The processor configured to estimate effective capacity by applying the one or more exhaust mass flow functions to the measured exhaust mass flow.

Some implementations of the present technology may include apparatus. The apparatus may include means for accessing a measured exhaust mass flow of a sieve bed for a purge phase of a pressure swing adsorption cycle of an oxygen concentrator, where the pressure in the sieve bed may be regulated to maintain a predetermined target pressure by adjusting the speed of a motor of a compression system of the oxygen concentrator. The apparatus may include means for accessing one or more exhaust mass flow functions. The apparatus may include means for estimating effective capacity by applying the one or more exhaust mass flow functions to the exhaust mass flow.

Some implementations of the present technology may include a method of estimating effective capacity of a canister system in an oxygen concentrator. The method may include accessing a measured motor speed of a motor of a compression system of the oxygen concentrator with a predetermined target pressure, wherein the pressure in the canister system of the oxygen concentrator may be regulated to maintain a predetermined target pressure by adjusting the speed of the motor. The method may include accessing one or more motor speed functions. The method may include estimating the effective capacity of the canister system by applying the one or more motor speed functions to the measured motor speed.

The one or more motor speed functions may use a fresh value of the motor speed. The fresh value may be a value of the motor speed obtained from a fresh sieve bed of a same type as the sieve bed at the predetermined target pressure. The one or more motor speed functions may use a fully degraded value of the motor speed. The fully degraded value may be a value of the motor speed obtained from a fully degraded sieve bed of a same type as the sieve bed at the predetermined target pressure. The one or more motor speed functions may comprise an interpolation using the fresh value of the motor speed and the fully degraded value of the motor speed. The method may include repeating the accessing and the estimating to obtain a further estimate of effective capacity. The method may include estimating a remaining usage time of the sieve bed from the estimate and the further estimate of effective capacity. The method may include displaying, on a display of the oxygen concentrator, an indicator of the estimated effective capacity. The method may include generating a message based on the estimated effective capacity.

Some implementations of the present technology may include an oxygen concentrator. The oxygen concentrator may include a canister system may include a sieve bed containing a gas separation adsorbent. The oxygen concentrator may include a canister system may include a sieve bed containing a gas separation adsorbent a compression system configured to feed a feed gas into the sieve bed. The oxygen concentrator may include a canister system may include a sieve bed containing a gas separation adsorbent. The oxygen concentrator may include a memory. The oxygen concentrator may include a controller. The controller may be configured to access a measured motor speed of a motor of a compression system of the oxygen concentrator with a predetermined target pressure, where the pressure in the canister system of the oxygen concentrator may be regulated to maintain a predetermined target pressure by adjusting the speed of the motor. The controller may be configured to access one or more motor speed functions. The controller may be configured to estimate effective capacity of the canister system by applying the one or more motor speed functions to the measured motor speed.

Some implementations of the present technology may include connected oxygen therapy system. The system may include a portable oxygen concentrator may include a canister system with a sieve bed containing a gas separation adsorbent. The system may include an external computing device in communication with the portable oxygen concentrator. The system may include memory. The system may include a processor configured by program instructions stored in the memory to estimate effective capacity of the sieve bed. The processor may be configured to access a measured motor speed of a motor of a compression system of the oxygen concentrator with a predetermined target pressure, where the pressure in the canister system of the oxygen concentrator may be regulated to maintain a predetermined target pressure by adjusting the speed of the motor. The processor may be configured to access one or more motor speed functions. The processor may be configured to estimate effective capacity of the canister system by applying the one or more motor speed functions to the measured motor speed.

Some implementations of the present technology may include apparatus. The apparatus may include means for accessing a measured motor speed of a motor of a compression system of an oxygen concentrator with a predetermined target pressure, wherein the pressure in a canister system of the oxygen concentrator may be regulated to maintain a predetermined target pressure by adjusting the speed of the motor. The apparatus may include means for accessing one or more motor speed functions of the motor speed. The apparatus may include means for estimating effective capacity of the canister system by applying the one or more motor speed functions to the measured motor speed.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### BRIEF DESCRIPTION OF THE DRAWINGS

Advantages of the present technology will become apparent to those skilled in the art with the benefit of the following detailed description of implementations and upon reference to the accompanying drawings in which similar reference numerals indicate similar components:

FIG. 1A depicts an oxygen concentrator in accordance with one form of the present technology.

FIG. 1B is a schematic diagram of the gas separation system of the oxygen concentrator of FIG. 1A.

FIG. 1C is a side view of the main components of the oxygen concentrator of FIG. 1A.

FIG. 1D is a perspective side view of a compression system of the oxygen concentrator of FIG. 1A.

FIG. 1E is a side view of a compression system that includes a heat exchange conduit.

FIG. 1F is a schematic diagram of example outlet components of the oxygen concentrator of FIG. 1A.

FIG. 1G depicts an outlet conduit for the oxygen concentrator of FIG. 1A.

FIG. 1H depicts an alternate outlet conduit for the oxygen concentrator of FIG. 1A.

FIG. 1I is a perspective view of a disassembled canister system for the oxygen concentrator of FIG. 1A.

FIG. 1J is an end view of the canister system of FIG. 1I.

FIG. 1K is an assembled view of the canister system end depicted in FIG. 1J.

FIG. 1L is a view of an opposing end of the canister system of FIG. 1I to that depicted in FIGS. 1J and 1K.

FIG. 1M is an assembled view of the canister system end depicted in FIG. 1L.

FIG. 1N depicts an example control panel for the oxygen concentrator of FIG. 1A.

FIG. 1O depicts a connected oxygen therapy system that includes one or more oxygen concentrators, such as the oxygen concentrator 100 of FIG. 1A.

FIG. 2 is an illustration of one complete PSA cycle of a PSA process according to one implementation of the present technology.

FIG. 3 is a schematic diagram of a motor control circuit according to one implementation of the present technology.

FIG. 4 illustrates a state machine used to implement a PSA cycle according to one implementation of the present technology.

FIGS. 5A and 5B contain graphs illustrating relationships between an operational parameter of a sieve bed and the effective capacity of the sieve bed.

FIGS. 6A and 6B illustrate models of gas flows in a gas separation system such as the gas separation system of FIG. 1B.

FIG. 7 contains a flow chart illustrating a method of estimating the effective capacity of the sieve beds of a POC in an effective capacity measurement mode according to one implementation of the present technology.

FIG. 8 contains a flow chart illustrating a method of estimating the effective capacity of the sieve beds of a POC in an effective capacity measurement mode according to one implementation of the present technology.

FIG. 9 contains a flow chart illustrating a method of estimating the effective capacity of the sieve beds of a POC in an effective capacity measurement mode according to one implementation of the present technology.

FIG. 10 contains a flow chart illustrating a method of estimating the effective capacity of the sieve beds of a POC during normal operation of the fine pressure regulation mode according to one implementation of the present technology.

FIG. 11 contains a flow chart illustrating a method of estimating the effective capacity of the sieve beds of a POC during normal operation according to one implementation of the present technology.

FIG. 12 contains a flow chart illustrating a method of characterising a compressor of a POC in a compressor characterisation mode according to one implementation of the present technology.

FIG. 13 contains a flow chart illustrating a method of characterising a compressor of a POC in a compressor characterisation mode according to one implementation of the present technology.

### DETAILED DESCRIPTION OF THE IMPLEMENTATIONS

Aspects of the present technology are described in detail with reference to the drawing figures wherein like reference numerals identify similar or identical elements. It is to be understood that the disclosed implementation are merely examples of the technology, which may be implemented in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present technology in virtually any appropriately detailed structure.

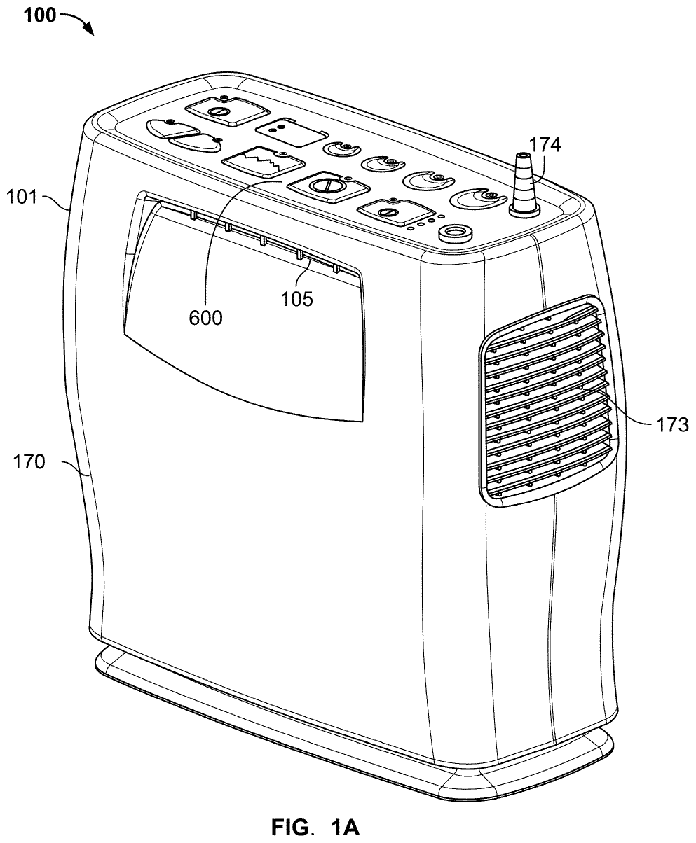

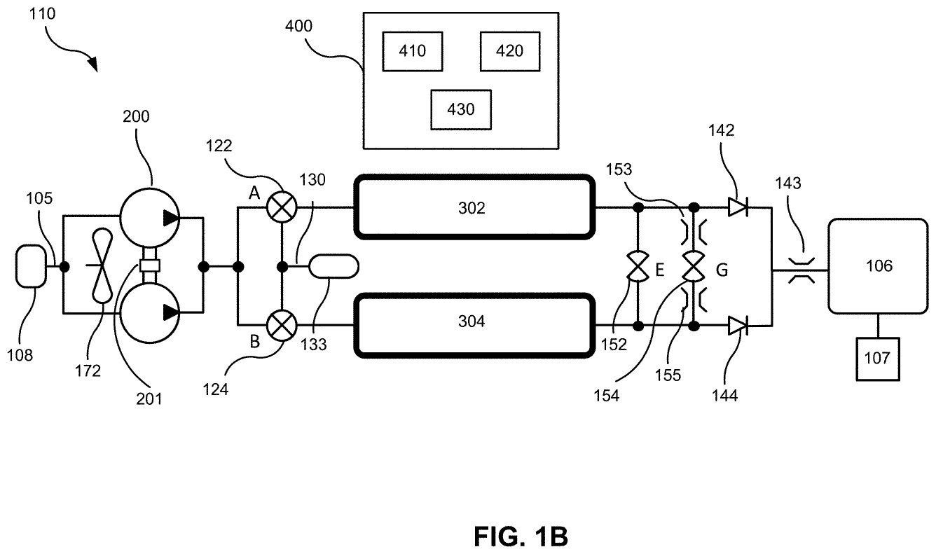

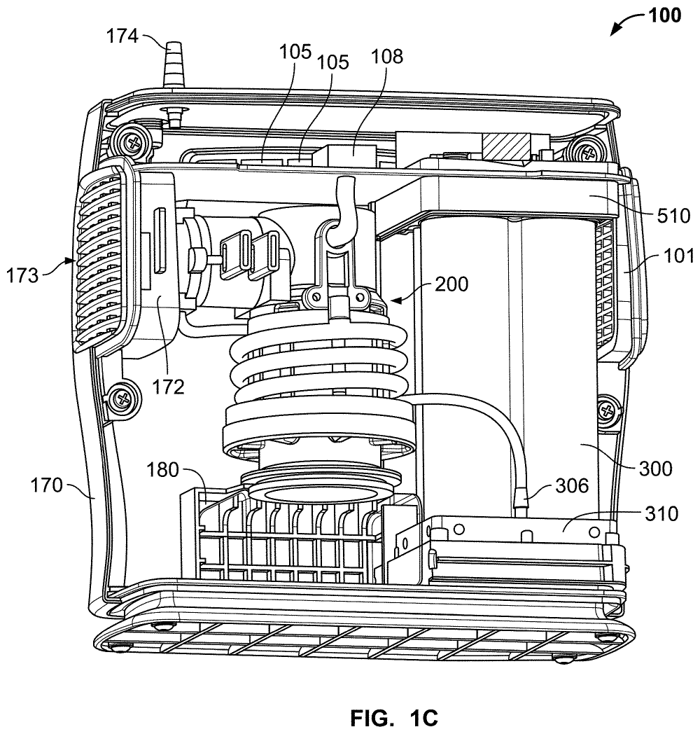

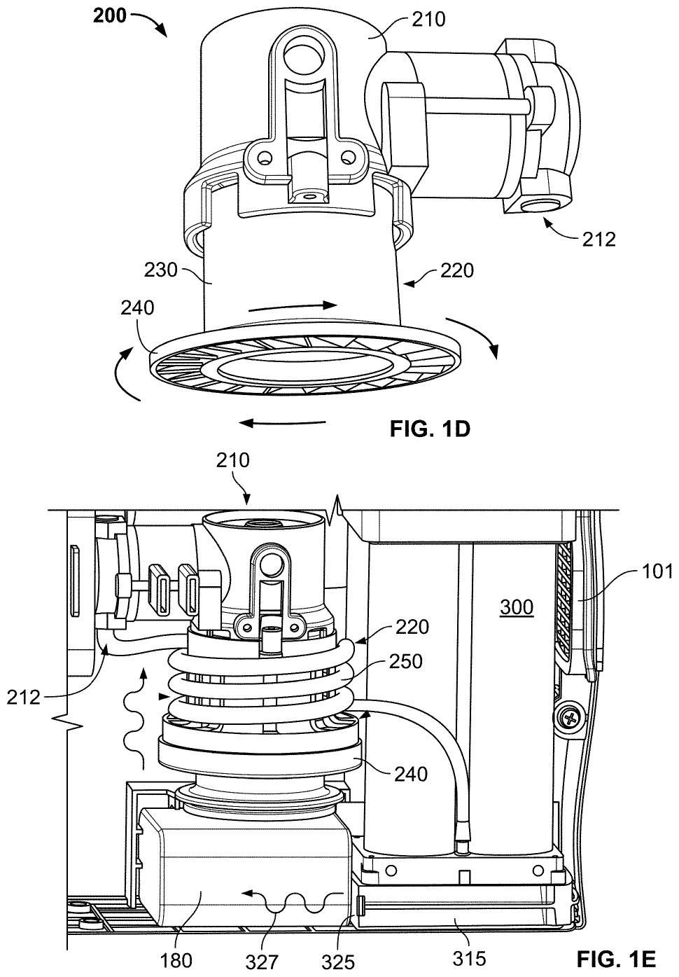

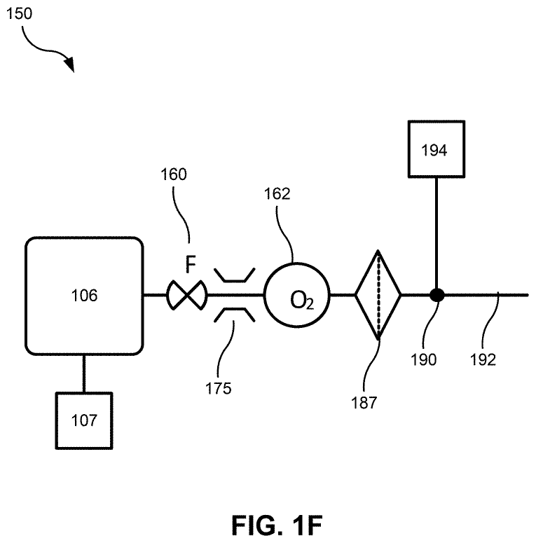

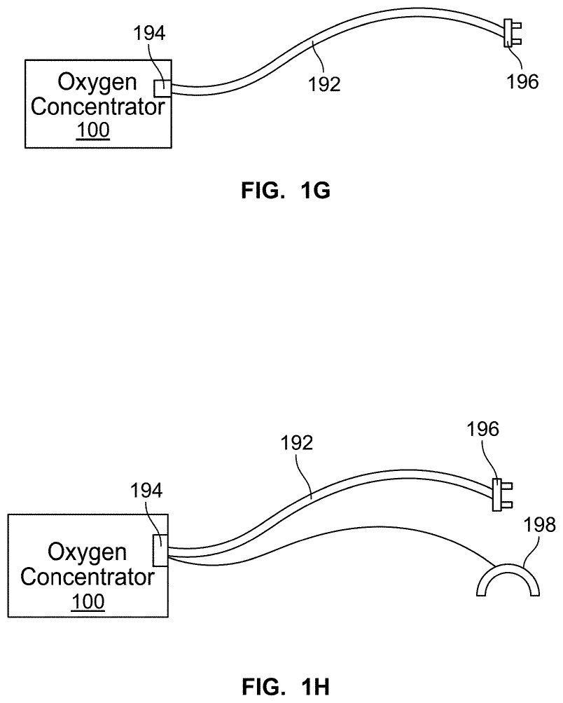

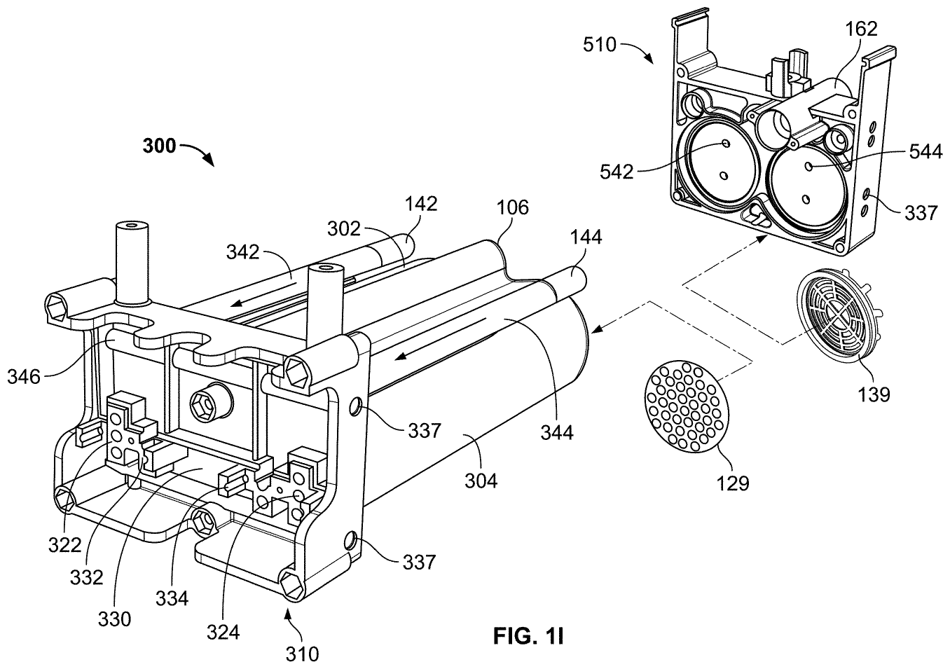

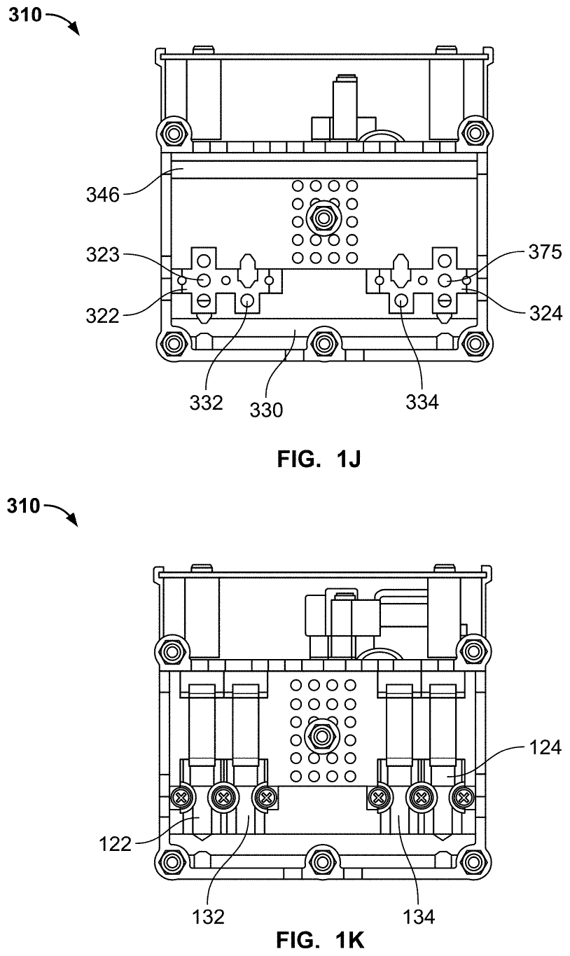

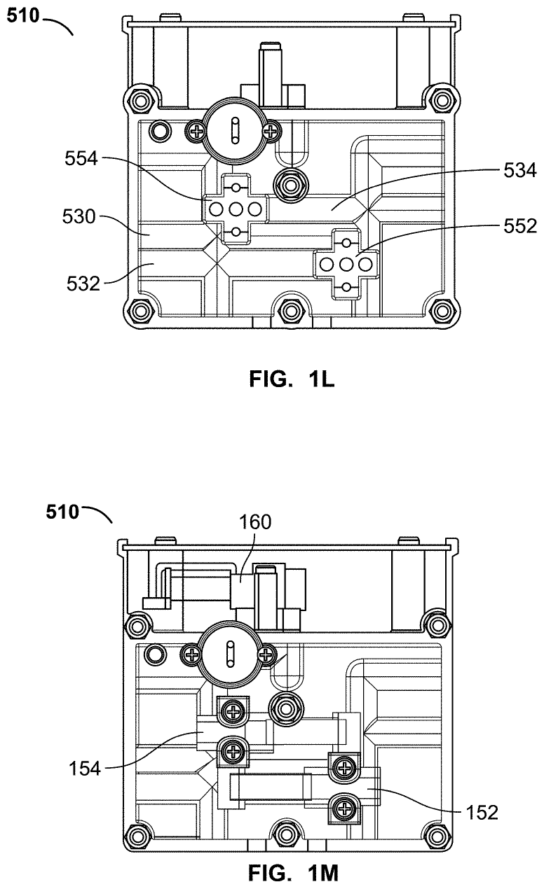

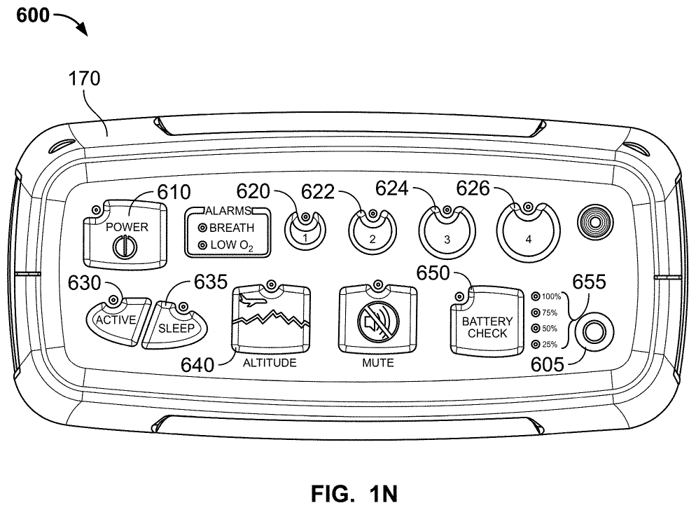

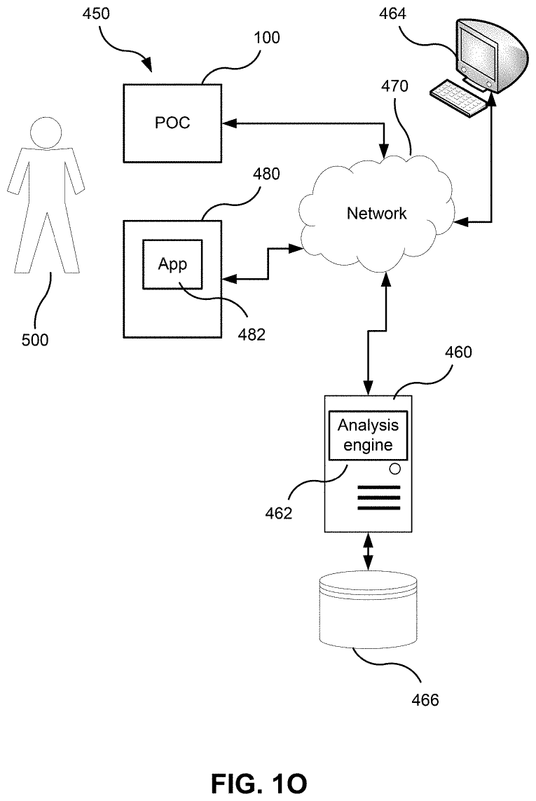

FIGS. 1A to 1N illustrate an implementation of an oxygen concentrator 100. Oxygen concentrator 100 may concentrate oxygen within an air stream to provide oxygen enriched air to a user. Oxygen concentrator 100 may be a portable oxygen concentrator. For example, oxygen concentrator 100 may have a weight and size that allows the oxygen concentrator to be carried by hand and/or in a carrying case. In one implementation, oxygen concentrator 100 has a weight of less than about 20 pounds, less than about 15 pounds, less than about 10 pounds, or less than about 5 pounds. In an implementation, oxygen concentrator 100 has a volume of less than about 1000 cubic inches, less than about 750 cubic inches, less than about 500 cubic inches, less than about 250 cubic inches, or less than about 200 cubic inches.

As described herein, oxygen concentrator 100 uses a pressure swing adsorption (PSA) process (which is cyclic) to produce oxygen enriched air. However, in other implementations, oxygen concentrator 100 may be modified such that it uses a cyclic vacuum swing adsorption (VSA) process or a cyclic vacuum pressure swing adsorption (VPSA) process to produce oxygen enriched air.

Oxygen concentrator 100 is configured as described in more detail below to deliver oxygen enriched air at one of multiple user-selectable flow rate settings (or flow settings), each of which corresponds to a flow rate of the delivered oxygen enriched air. In one implementation, there are six user-selectable flow rate settings. Table 1 contains example flow rates corresponding to each of the six flow rate settings, numbered 1 to 6. The flow rate values in Table 1 correspond to minute volumes (bolus volume in litres multiplied by breathing rate per minute) of delivered oxygen enriched gas in litres per minute (LPM).

TABLE 1
Example flow rates corresponding to each of six flow rate
settings in one implementation of the present technology.

| Flow rate setting | Flow rate (LPM) |
|---|---|
| 1 | 0.2 |
| 2 | 0.4 |
| 3 | 0.6 |
| 4 | 0.8 |
| 5 | 1.0 |
| 6 | 1.1 |

Outer Housing

FIG. 1A depicts an implementation of an outer housing 170 of an oxygen concentrator 100. In some implementations, outer housing 170 may be comprised of a light-weight plastic. Outer housing includes compression system inlets 105, cooling system passive inlet 101 and outlet 173 at each end of outer housing 170, outlet port 174, and control panel 600. Inlet 101 and outlet 173 allow cooling air to enter the housing, flow through the housing, and exit the interior of housing 170 to aid in cooling of the oxygen concentrator 100. Compression system inlets 105 allow air to enter the compression system. Outlet port 174 is used to attach a conduit to provide oxygen enriched air produced by the oxygen concentrator 100 to a user.

### Gas Separation System

FIG. 1B illustrates a schematic diagram of a gas separation system 110 of an oxygen concentrator such as the oxygen concentrator 100, according to an implementation. The gas separation system 110 may concentrate oxygen within an air stream to provide oxygen enriched air to an outlet system (described below).

Oxygen enriched air may be produced from ambient air by pressurising ambient air in canisters 302 and 304, which contain a gas separation adsorbent and are therefore referred to as sieve beds. Gas separation adsorbents useful in an oxygen concentrator are capable of separating at least nitrogen from an air stream to produce oxygen enriched air. Examples of gas separation adsorbents include molecular sieves that are capable of separating nitrogen from an air stream. Examples of adsorbents that may be used in an oxygen concentrator include, but are not limited to, zeolites (natural) or synthetic crystalline aluminosilicates that separate nitrogen from an air stream under elevated pressure. Examples of synthetic crystalline aluminosilicates that may be used include, but are not limited to: OXYSIV adsorbents available from UOP LLC, Des Plaines, Ill.; SYLOBEAD adsorbents available from W. R. Grace & Co, Columbia, Md.; SILIPORITE adsorbents available from CECA S.A. of Paris, France; ZEOCHEM adsorbents available from Zeochem AG, Uetikon, Switzerland; and AgLiLSX adsorbent available from Air Products and Chemicals, Inc., Allentown, Pa.

As shown in FIG. 1B, air may enter the gas separation system 110 through air inlet 105. Air may be drawn into air inlet 105 by compression system 200. Compression system 200 may draw in air from the surroundings of the oxygen concentrator and compress the air, forcing the compressed air into one or both canisters 302 and 304. In an implementation, an inlet muffler 108 may be coupled to air inlet 105 to reduce sound produced by air being pulled into the oxygen concentrator by compression system 200. In an implementation, inlet muffler 108 may reduce moisture and sound. For example, a water adsorbent (desiccant) material (such as a polymer water adsorbent material or a zeolite material) may be used to both adsorb water from the incoming air and to reduce the sound of the air passing into the air inlet 105. In an implementation, inlet muffler 108 may reduce contaminant particles (dust) and sound. For example, a dust filter may be used to both remove dust from the incoming air and to reduce the sound of the air passing into the air inlet 105.

Compression system 200 may include one or more compressors configured to compress air. Pressurized air, produced by compression system 200, may be fed into one or both of the canisters 302 and 304.

Coupled to each canister 302/304 are valves such as three-way inlet valves 122/124. As shown in FIG. 1B, inlet valve 122 (labelled A) is coupled to the “feed end” of canister 302 and inlet valve 124 (labelled B) is coupled to the “feed end” of canister 304. Inlet valves 122/124 are used to control the passage of air from compression system 200 to the respective canisters, and to vent exhaust gas from the respective canisters to atmosphere. In some implementations, inlet valves 122/124 may be silicon plunger solenoid valves. Other types of valves, however, may be used, such as poppet valves or piezoelectric valves. Plunger valves offer advantages over other kinds of valves by being quiet and having low slippage. In some implementations, one or both inlet valves 122/124 may be replaced by pairs of two-way valves that actuate in antiphase to emulate a three-way valve.

In some implementations, a two-step valve actuation voltage may be generated to control inlet valves 122/124. For example, a high voltage (e.g., 24 V) may be applied to an inlet valve to actuate the inlet valve. The voltage may then be reduced (e.g., to 7 V) to keep the inlet valve actuated. Using less voltage to keep a valve open may use less power. This reduction in voltage minimizes heat buildup and power consumption to extend run time from the internal power supply 180 (described below). When the power is cut off to an inlet valve 122/124, the valve de-actuates by spring action. In some implementations, the voltage may be applied as a function of time that is not necessarily a stepped response (e.g., a curved downward voltage between an initial 24 V and a final 7 V).

In an implementation, a controller 400 is electrically coupled to inlet valves 122 and 124 by an input/output interface. Controller 400 includes one or more processors 410 operable to execute program instructions stored in memory 420. The program instructions configure the controller to perform various predefined methods that are used to operate the oxygen concentrator, such as the methods described in more detail herein. The program instructions may include program instructions for generating control signals via the output interface to operate inlet valves 122 and 124 out of phase with each other, i.e., when one of inlet valves 122 or 124 is actuated, the other valve is de-actuated. In some implementations, the voltages and the durations of the voltages used to actuate the inlet valves 122 and 124 may be controlled by controller 400. The controller 400 may also include a transceiver 430 that may communicate with external devices to transmit data collected by the processor 410 or receive instructions from an external device for the processor 410.

Check valves 142 and 144 are coupled to the “product ends” of canisters 302 and 304, respectively. Check valves 142 and 144 may be one-way valves that are passively operated by the pressure differentials that occur as the canisters are pressurized and vented, or may be active valves. Check valves 142 and 144 are coupled to the canisters to allow oxygen enriched air produced during pressurization of each canister to flow out of the canister, and to inhibit back flow of oxygen enriched air or any other gases into the canister. In this manner, check valves 142 and 144 act as one-way valves allowing oxygen enriched air to exit the respective canisters during pressurization.

The term “check valve”, as used herein, refers to a valve that allows flow of a fluid (gas or liquid) in one direction and inhibits back flow of the fluid. The term “fluid” may include a gas or a mixture of gases (such as air). Examples of check valves that are suitable for use include, but are not limited to: a ball check valve; a diaphragm check valve; a butterfly check valve; a swing check valve; a duckbill valve; an umbrella valve; and a lift check valve. Under pressure, nitrogen molecules in the pressurized ambient air are adsorbed by the gas separation adsorbent in the pressurized canister. As the pressure increases, more nitrogen is adsorbed until the gas in the canister is enriched in oxygen. The nonadsorbed gas molecules (mainly oxygen) flow out of the pressurized canister when the pressure reaches a point sufficient to overcome the resistance of the check valve coupled to the canister. In one implementation, the pressure drop of the check valve in the forward direction is less than 1 psi. The break pressure in the reverse direction is greater than 100 psi. It should be understood, however, that modification of one or more components would alter the operating parameters of these valves. If the forward flow pressure drop of the check valve is increased, there is, generally, a reduction in oxygen enriched air production. If the break pressure for reverse flow is reduced or set too low, there is, generally, a reduction in oxygen enriched air pressure.

In an implementation, pressurized air is fed into one of canisters 302 or 304 while the other canister is being vented. For example, during use, inlet valve 122 is de-actuated while inlet valve 124 is actuated. Pressurized air from compression system 200 is fed into canister 302 via the de-actuated inlet valve 122, while being inhibited from entering canister 304 by the actuated inlet valve 124. During pressurization of canister 302, actuated inlet valve 124 connects canister 304 to atmosphere to allow exhaust gas (mainly nitrogen) to vent from canister 304 to atmosphere through concentrator exhaust outlet 130. In an implementation, the exhaust gas may be directed through muffler 133 to reduce the noise produced by venting the exhaust gas from the canister. As exhaust gas is vented from canister 304, the pressure in the canister 304 drops, allowing the nitrogen to become desorbed from the gas separation adsorbent. The desorption of the nitrogen leaves canister 304 in a state that allows renewed separation of nitrogen from a pressurised air stream. Muffler 133 may include open cell foam (or another material) to muffle the sound of the exhaust gas leaving the oxygen concentrator. In some implementations, the combined muffling components/techniques for the input of air and the output of oxygen enriched air may provide for oxygen concentrator operation at a sound level below 50 decibels.

After some time, the pressure in canister 302 is sufficient to open check valve 142. Oxygen enriched air produced in canister 302 passes through check valve 142 and flow restrictor 143 and, in one implementation, is collected in accumulator 106. The flow restrictor 143 controls the flow of oxygen enriched air to the accumulator 106. For example, when the accumulator 106 is depressurised upon bolus release (described below), if flow restrictor 143 is not present (and the intervening path has very low impedance), accumulator 106 draws gas at a high flow rate from the canister currently in pressurisation or adsorption. As a result, the pressure in the canister significantly drops, which tends to draw un-enriched air to the accumulator 106, thereby reducing the oxygen concentration. Additionally, gas exchange between sieve beds via the E- and G-valves 152 and 154 (described below) to maintain high oxygen oxygen concentration at the product end of the canisters will be significantly affected causing disruption in the overall PSA cycle. The presence of the flow restrictor 143 helps to replace released oxygen enriched air at an optimal rate and damp the above detrimental effects.

After some further time, the gas separation adsorbent in canister 302 becomes saturated with nitrogen and is unable to separate significant amounts of nitrogen from incoming air. In the implementation described above, when the gas separation adsorbent in canister 302 reaches this saturation point, which may be inferred to have occurred after a predetermined interval, a two-way valve 152 (labelled E) is actuated, which directly connects canister 302 to 304 at their product ends. This causes the pressure in canister 302 to fall rapidly while the pressure in canister 304 rises equally rapidly towards equilibrium with canister 302. Inlet valve 124 is then de-actuated, connecting compression system 200 to canister 304 to assist with this equalisation of pressures from the feed end. Once the pressures in the canisters are equalised, which may be inferred to have occurred after a predetermined interval, valve 152 is de-actuated to isolate the canisters once again, and inlet valve 122 is actuated, stopping the feed of compressed air to canister 302 and connecting canister 302 to atmosphere to allow venting of exhaust gas. While canister 302 is being vented, canister 304 is pressurized to produce oxygen enriched air in the same manner described above. Pressurization of canister 304 is achieved through de-actuated inlet valve 124. After a time, the oxygen enriched air exits canister 304 through check valve 144.

During venting of the canisters, it is advantageous that at least a majority of the nitrogen is removed. In an implementation, at least about 50%, at least about 60%, at least about 70%, at least about 80%, at least about 90%, at least about 95%, at least about 98%, or substantially all of the nitrogen in a canister is removed before the canister is re-used to separate nitrogen from air.

In some implementations, nitrogen removal may be assisted using an oxygen enriched air stream that is introduced into the canister from the other canister or stored oxygen enriched air. In an exemplary implementation, a portion of the oxygen enriched air may be transferred from canister 302 to canister 304 when canister 304 is being vented of exhaust gas. Transfer of oxygen enriched air from canister 302 to canister 304 during venting of canister 304 helps to desorb nitrogen from the adsorbent by lowering the partial pressure of nitrogen adjacent the adsorbent. The flow of oxygen enriched air also helps to purge desorbed nitrogen (and other gases) from the canister. In an implementation, oxygen enriched air may travel through flow restrictors 153 and 155 between the two canisters. Flow restrictors 153 and 155 may be 0.63 mm diameter flow restrictors. Other flow restrictor types and sizes are also contemplated and may be used depending on the specific configuration and tubing used to couple the canisters. In some implementations, the flow restrictors 153 and 155 may be press fit flow restrictors that restrict air flow by introducing a narrower diameter in their respective conduits. In some implementations, the press fit flow restrictors may be made of sapphire, metal or plastic (other materials are also contemplated).

Flow of oxygen enriched air between the canisters is also controlled by use of a two-way valve 154 (labelled G). The G-valve 154 may be opened during the venting process and may be closed otherwise to prevent excessive oxygen loss out of the purging canister. Other durations are also contemplated. In an exemplary implementation, canister 302 is being vented and it is desirable to purge canister 302 by passing a portion of the oxygen enriched air being produced in canister 304 into canister 302. A portion of oxygen enriched air is passed into canister 302, from canister 304, through valve 154 and flow restrictors 153 and 155. The selection of appropriate flow restrictors 153 and 155, coupled with controlled opening of valve 154 allows a controlled amount of oxygen enriched air to be passed from canister 304 to purge canister 302. In an implementation, the controlled amount of oxygen enriched air is an amount sufficient to purge canister 302 and minimize the loss of oxygen enriched air to atmosphere through valve 122 of canister 302. While this implementation describes purging of canister 302, it should be understood that the same process can be used to purge canister 304 using valve 154 and flow restrictors 153 and 155.

Valve 154 works with flow restrictors 153 and 155 to optimize the gas flow balance between the two canisters. This may allow for better flow control for purging one of the canisters with oxygen enriched air from the other of the canisters. It may also provide better flow direction between the two canisters. In some implementations, the purge flow pathway may not have flow restrictors but instead the G-valve may have built-in resistance to flow, or the purge flow pathway itself may have a narrow radius to provide flow resistance.

In some implementations, the purge flow is stopped by de-actuating valve 154 at the same time as valve 152 is de-actuated, to complete the isolation of the two canisters when the pressures therein are equalised.

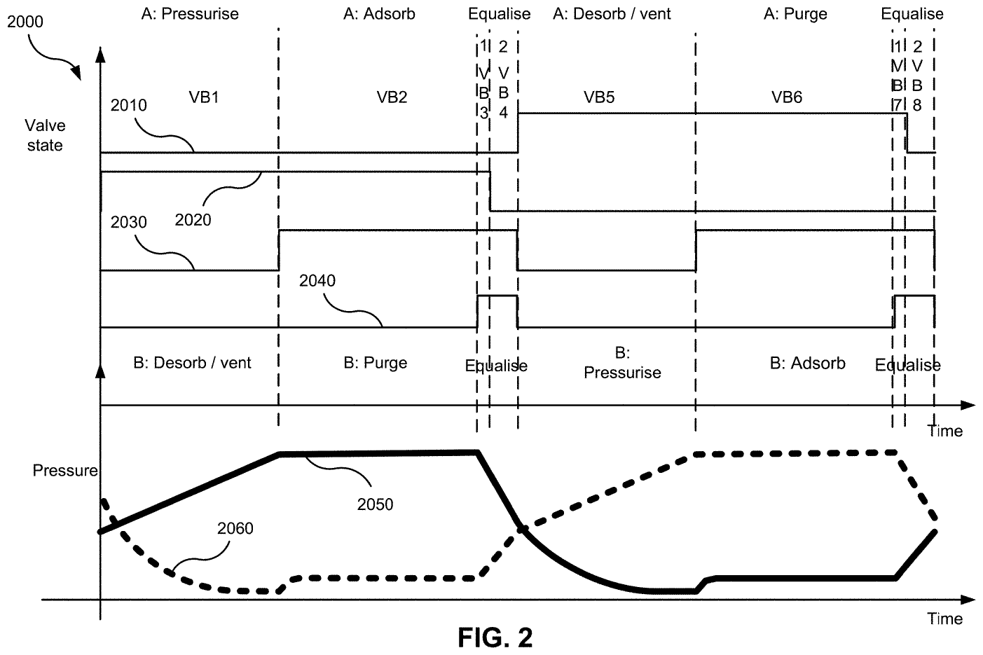

FIG. 2 is an illustration of one complete PSA cycle 2000 of a PSA process according to one implementation of the present technology. FIG. 2 contains valve actuation waveforms 2010, 2020, 2030, and 2040 for the A-, B-, E-, and G-valves 122, 124, 152, and 154 respectively, that represent valve control signals generated by the controller 400. FIG. 2 also contains pressure waveforms 2050 and 2060 indicative of the pressure in canisters 302 and 304 synchronised with the waveforms 2010, 2020, 2030, and 2040.

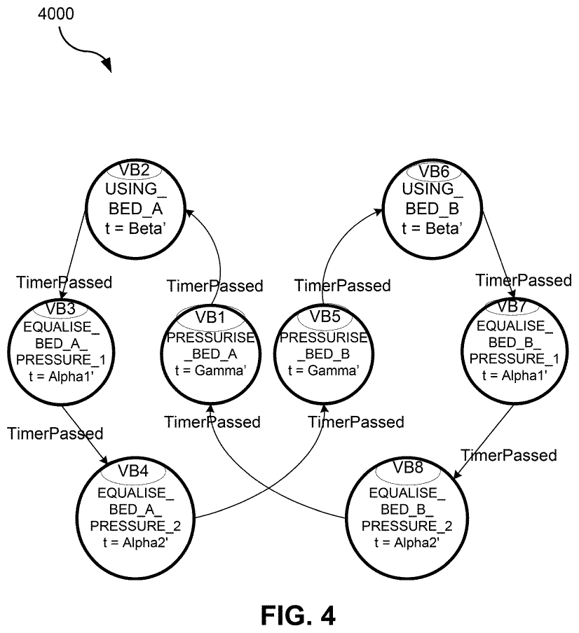

The PSA cycle 2000 illustrated in FIG. 2 contains eight sequential phases, each phase corresponding to a particular set of valve states (actuated or de-actuated) (e.g., respectively associated with high and low states of the signal of the waveform). The PSA cycle 2000 will be described with reference to the PSA state machine 4000 illustrated in FIG. 4.

The PSA cycle 2000 starts with the state machine 4000 entering the state labelled VB1 (“PRESSURIZE_BED_A”). On entering state VB1, a timer is set to a duration labelled Gamma′. State machine 4000 remains in state VB1 for a first phase which ends when the timer times out. During the first phase, canister 302 (labelled A) is being pressurised by the de-actuation of the A-valve 122. The pressure waveform 2050 indicates a steady rise in the pressure of canister 302. Canister 302 starts to adsorb nitrogen and produce oxygen enriched air. Oxygen enriched air exits the canister 302 when the check valve 142 opens. Meanwhile canister 304 (labelled B) is being vented by the actuation of the B-valve 124. The pressure waveform 2060 indicates a rapid depressurisation of canister 304. The first phase is therefore referred to as the pressurisation phase of canister 302 and the desorb/vent phase of canister 304. The E- and G-valves 152 and 154 are de-actuated to prevent any interchange of gas between the canisters at their product ends.

When the timer times out, the state machine 4000 transitions to the state labelled VB2 (“USING_BED_A”). On entering state VB2, the timer is set to a duration labelled Beta′, and the G-valve 154 is actuated, allowing a portion of the oxygen enriched air leaving canister 302 to purge desorbed nitrogen and other gases from canister 304. State machine 4000 remains in state VB2 for a second phase which ends when the timer times out. The pressure waveform 2050 indicates that the pressure in canister 302 stabilises, and canister 302 continues to adsorb nitrogen and produce oxygen enriched air. Meanwhile the pressure waveform 2060 indicates that the pressure in canister 304 rises a little, and then stabilises. The second phase is therefore referred to as the adsorption phase for canister 302, and the purge phase for canister 304.

When the timer times out, the state machine 4000 transitions to the state labelled VB3 (“EQUALISE_BED_A_PRESSURE_1”). On entering state VB3, the timer is set to a duration labelled Alpha1′, and the E-valve 152 is actuated, which directly connects canister 302 to 304 at their product ends. This causes the pressure in canister 302 to fall rapidly, as the pressure waveform 2050 indicates, while the pressure in canister 304 rises equally rapidly towards equilibrium with canister 302, as the pressure waveform 2060 indicates. State machine 4000 remains in state VB3 for a third phase which ends when the timer times out. The third phase is referred to as the equalisation (1) phase for canister 302.

When the timer times out, the state machine 4000 transitions to the state labelled VB4 (“EQUALISE_BED_A_PRESSURE_2”). On entering state VB4, the timer is set to a duration labelled Alpha2′, and the B-valve 124 is de-actuated, ending the venting of canister 304 and connecting compression system 200 to canister 304 to assist with this equalisation of pressures from the feed end. The pressure waveforms 2050 and 2060 indicate that the pressures in canisters 302 and 304 continue to fall and rise respectively. State machine 4000 remains in state VB4 for a fourth phase which ends when the timer times out. The fourth phase is referred to as the equalisation (2) phase for canister 302.

When the timer times out, the state machine 4000 transitions to the state labelled VB5 (“PRESSURIZE_BED_B”). On entering state VB5, the timer is set to Gamma′, and the A-valve 122 is actuated to disconnect canister 302 from the compression system 200 and connect canister 302 to atmosphere to allow exhaust gas to vent. Simultaneously, the G-valve 154 and the E-valve 152 are de-actuated to prevent any interchange of gas between the canisters at their product ends. The B-valve 124 remains de-actuated. State machine 4000 remains in state VB5 for a fifth phase which ends when the timer times out. The pressure waveform 2050 indicates a continued depressurisation of canister 302 towards ambient pressure. The pressure waveform 2060 indicates a steady rise in the pressure of canister 304. Canister 304 starts to adsorb nitrogen and produce oxygen enriched air. Oxygen enriched air exits the canister 304 when the check valve 144 opens. The fifth phase therefore mirrors the first phase with the roles of canisters 302 and 304 reversed and is therefore referred to as the pressurisation phase of canister 304 and the desorb/vent phase of canister 302.

When the timer times out, the state machine 4000 transitions to the state labelled VB6 (“USING_BED_B”). On entering state VB6, the timer is set to Beta′, and the G-valve 154 is actuated, allowing a portion of the oxygen enriched air leaving canister 304 to purge desorbed nitrogen and other gases from canister 302. State machine 4000 remains in state VB6 for a sixth phase which ends when the timer times out. The pressure waveform 2050 indicates that the pressure in canister 302 rises a little, and then stabilises. Meanwhile the pressure waveform 2060 indicates that the pressure in canister 304 stabilises as canister 304 continues to adsorb nitrogen and produce oxygen enriched air. The sixth phase is referred to as the purge phase for canister 302, and the adsorption phase for canister 304.

When the timer times out, the state machine 4000 transitions to the state labelled VB7 (“EQUALISE_BED_B_PRESSURE_1”). On entering state VB7, the timer is set to Alpha1′, and the E-valve 152, which directly connects canister 302 to 304 at their product ends, is actuated. This causes the pressure in canister 302 to rise rapidly, as the pressure waveform 2050 indicates, while the pressure in canister 304 falls equally rapidly towards equilibrium with canister 304, as the pressure waveform 2060 indicates. State machine 4000 remains in state VB7 for a seventh phase which ends when the timer times out. The seventh phase is therefore referred to as the equalisation (1) phase for canister 304.

When the timer times out, the state machine 4000 transitions to the state labelled VB8 (“EQUALISE_BED_B_PRESSURE_2”). On entering state VB8, the timer is set to Alpha2′, and the A-valve 122 is de-actuated, ending the venting of canister 302 and connecting compression system 200 to canister 302 to assist with this equalisation of pressures from the feed end. The pressure waveforms 2050 and 2060 indicate that the pressures in canisters 302 and 304 continue to rise and fall respectively. The eighth phase is therefore referred to as the equalisation (2) phase for canister 304. When the timer times out, the PSA cycle 2000 is complete and the state machine 4000 returns to state VB1 to start another PSA cycle.

The first to fourth phases make up a PSA half cycle while the fifth to eighth phases make up the other PSA half cycle.

Table 2 contains example timer settings, referred to as base phase durations, in milliseconds, for each phase of a PSA cycle in each of six flow rate settings according to one implementation of the present technology.

TABLE 2
Base phase durations, in milliseconds, for a PSA half cycle
at each of six flow rate settings

| | Gamma′ | Beta′ | Alpha1′ | Alpha2′ |
|---|---|---|---|---|
| Flow | (pressurisation | (adsorption | (Equalisation | (Equalisation |
| rate | phases 1 | phases 2 | (1) phases | (2) phases |
| setting | and 5) | and 6) | 3 and 7) | 4 and 8) |
| 1 | 2800 | 2800 | 100 | 500 |
| 2 | 2600 | 2600 | 100 | 500 |
| 3 | 2500 | 2500 | 100 | 500 |
| 4 | 2300 | 2300 | 100 | 500 |
| 5 | 2100 | 2100 | 100 | 500 |
| 6 | 2100 | 2100 | 100 | 500 |

The pressurisation and adsorption phase durations in Table 2 become shorter with increasing flow rate setting, which may appear counter-intuitive. However, the shortening phase durations with increasing flow rate setting are more than compensated for by increasing compressor output, as described in more detail below.

In some implementations, the phase durations for a full PSA cycle are identical (and equal to the base phase durations) between the two PSA half cycles. However, in some implementations, there are differences in the phase durations between the two half cycles of a full PSA cycle. Table 3 contains static adjustments to the base durations of each phase (applied across all flow rate settings) in one implementation of the present technology.

TABLE 3
Static adjustments to phase durations of each
PSA cycle phase, in milliseconds.

| Phase | Static adjustment (ms) |
|---|---|
| 1 | 0 |
| 2 | 0 |
| 3 | 20 |
| 4 | 0 |
| 5 | 0 |
| 6 | 0 |
| 7 | 0 |
| 8 | 0 |

Static adjustments may be predetermined based on knowledge of fixed asymmetries between the pneumatic paths associated with canisters 302 and 304. Such asymmetries may arise because of impedance differences between the flow paths including each canister as a result of manufacturing tolerances. For example, the static adjustment to the duration of phase 3 (the equalisation (1) phase of canister 302) according to Table 3 is 20 ms. This means that phase 3 is statically 20 ms longer than the base duration of phases 3 and 7. Since the static adjustment to the duration of phase 7 is 0, this means that phase 3 is statically 20 ms longer than phase 7. Using the base phase duration values in Table 2, this means that the static durations of phase 3 and phase 7 are 120 ms and 100 ms respectively. Such a difference counteracts an asymmetry in the E-valve 152, which has higher impedance in the direction from canister 302 to 304 than it does in the direction from canister 304 to 302. The equalisation (1) phase for canister 302 therefore needs to be slightly longer to achieve the same volume of equalisation flow from canister 302 to canister 304 as from canister 304 to canister 302 during the equalisation (1) phase of canister 304.

Compression System

Referring to FIG. 1C, an implementation of an oxygen concentrator 100 is depicted. Oxygen concentrator 100 includes a compression system 200, a canister system 300, and an internal power supply 180 disposed within an outer housing 170. Inlets 101 are located in outer housing 170 to allow air from the environment to enter oxygen concentrator 100. Inlets 101 may allow air to flow into the compartment to assist with cooling of the components in the compartment. Power supply 180, which may be a battery pack, provides power for the oxygen concentrator 100. Compression system 200 draws air in through the inlet 105 and muffler 108. As mentioned above, muffler 108 may reduce noise of air being drawn in by the compression system and also may include a desiccant material to remove water from the incoming air. Oxygen concentrator 100 may further include fan 172 used to vent air and other gases from the oxygen concentrator via outlet 173.

In some implementations, compression system 200 includes one or more compressors. In another implementation, compression system 200 includes a single compressor, coupled to all of the canisters of canister system 300. Turning to FIGS. 1D and 1E, a compression system 200 is depicted that includes compressor 210 and motor 220. Motor 220 is coupled to compressor 210 and provides an operating force to the compressor to operate the compression mechanism. For example, motor 220 may be a motor providing a rotatable component that causes cyclical motion of a component of the compressor that compresses air. When compressor 210 is a piston type compressor, motor 220 provides an operating force which causes the piston of compressor 210 to be reciprocated. Reciprocation of the piston causes compressed air to be produced by compressor 210. The pressure of the compressed air is, in part, estimated by the speed that the compressor is operated at, (e.g., how fast the piston is reciprocated). Motor 220, therefore, may be a variable speed motor that is operable at various speeds to dynamically control the pressure of air produced by compressor 210.

In one implementation, compressor 210 includes a single head wobble type compressor having a piston. Other types of compressors may be used such as diaphragm compressors and other types of piston compressors. Motor 220 may be a DC or AC motor and provides the operating power to the compressing component of compressor 210. Motor 220, in an implementation, may be a brushless DC motor. Motor 220 may be a variable speed motor configured to operate the compressing component of compressor 210 at variable speeds. Motor 220 may be coupled to controller 400, which sends operating signals to the motor to control the operation of the motor. For example, controller 400 may send signals to motor 220 to: turn the motor on, turn motor the off, and set the operating speed of the motor. Thus, as illustrated in FIG. 1B, the compression system 200 may include a speed sensor 201. The speed sensor 201 may be a motor speed transducer used to determine a rotational speed of the motor 220 and/or a frequency of another reciprocating operation of the compression system 200. For example, a motor speed signal from the motor speed transducer 201 may be provided to the controller 400. The speed sensor or motor speed transducer 201 may, for example, be a Hall effect sensor. The controller 400 may operate the compression system 200 via the motor 220 based on the speed signal and/or any other sensor signal of the oxygen concentrator 100, such as a pressure sensor (e.g., accumulator pressure sensor 107). Thus, the controller 400 receives sensor signals, such as a speed signal from the speed sensor 201 and an accumulator pressure signal from the accumulator pressure sensor 107. With such signal(s), the controller 400 may implement one or more control loops (e.g., feedback control) for operation of the compression system 200 based on sensor signals such as accumulator pressure and/or motor speed as described in more detail herein.

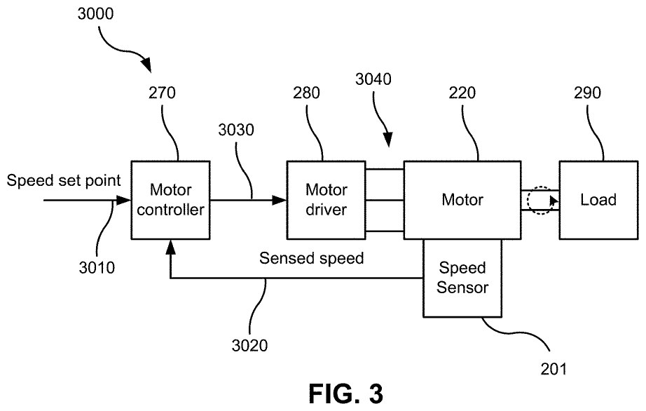

FIG. 3 is a schematic diagram of an example motor control circuit 3000 according to one implementation of the present technology in which the operating speed of the motor 220 is regulated to a speed set point while the motor 220 drives a load 290 including the compressor 210. Such speed control may be implemented by feedback control (closed-loop regulation). The size of the load 290 is representative of the back pressure experienced by the compressor 210 while it generates the pressurised air stream. The back pressure in turn is related to the pressure within whichever of the canisters 302 and 304 is being pressurised by the compressor 210.

In the motor control circuit 3000, a speed set point 3010 is provided to a motor controller 270, e.g. by the POC controller 400. The speed set point 3010 is obtained by the POC controller 400 as described in more detail below. The motor controller 270 may be implemented as an integrated circuit including, for example, one or more field programmable gate arrays (FPGAs), microcontrollers, etc. included on a circuit board disposed in the oxygen concentrator 100. Alternatively, the motor controller 270 may be implemented as part of the controller 400, configured by program instructions stored in internal memory 420 or an external memory medium coupled to controller 400, and executed by one or more processors 410.

The motor controller 270 also takes as input a speed signal 3020 from the speed sensor 201. The motor controller 270 processes the speed signal 3020 and the speed set point 3010 and generates a motor control signal 3030. The motor control signal 3030 is thus generated with a characteristic parameter that permits control of the motor 220 so as to drive the load 290 at the speed set point 3010. As long as the speed set point is fixed, the characteristic parameter of the motor control signal 3030 is representative of the size of the load 290 at any time. Since power is load multiplied by speed, which is roughly constant, the characteristic parameter is representative of the power being developed by the motor 220 and may be referred to as the power parameter.

As mentioned above, the load 290 is representative of the pressure within whichever of the canisters 302 and 304 is connected to the compressor 210 via its inlet valve 122 or 124. The power parameter of the motor control signal 3030 is therefore representative of the pressure within the canister currently connected to the compressor 210.

In one implementation, the motor control signal 3030 is a bi-valued (high or low) waveform consisting of a train of pulses at a predetermined frequency that is independent of the motor speed. In one implementation, the pulse frequency is 20 kHz. The duty cycle of the pulse train (ratio or proportion of high time during one period to the duration of one period) ranges between 0% (no pulses at all) and 100% (one continuous pulse). Such a waveform is referred to as a pulse-width modulation (PWM) waveform. The duty cycle of the PWM waveform is the power parameter of the PWM waveform. In this implementation, the motor controller 270 generates a PWM waveform with a duty cycle such that the motor 220 is able to drive the load 290 at the speed set point 3010. As long as the speed set point 3010 is fixed, the duty cycle of the PWM waveform at any time is therefore representative of the size of the load 290 at that time. The duty cycle of the PWM waveform (the power parameter of the motor control signal 3030) is therefore representative of the pressure within the canister currently connected to the compressor 210 via its inlet valve.

In other implementations, the motor control signal 3030 is a continuously-valued or discretely-valued DC signal such as a voltage or current. In such implementations, the power parameter may be the value of the motor control signal 3030 itself.

Returning to FIG. 3, the motor control signal 3030 is passed to a motor driver circuit 280 that generates one or more motor drive signals 3040. The motor driver circuit 280 may be implemented as an integrated circuit including, for example, one or more field programmable gate arrays (FPGAs), microcontrollers, etc. included on a circuit board disposed in the oxygen concentrator 100. The motor control signal 3030 modulates the motor drive signals 3040 to adjust the amount of power supplied to the motor 220 and hence the speed at which the motor 220 drives the load 290. In one example implementation, illustrated in FIG. 3, the motor 220 is a three-phase motor so there are three differently-phased motor drive signals 3040, one for each winding.

Heat Management

Compression system 200 inherently creates substantial heat. Heat is caused by the consumption of power by motor 220 and the losses and inefficiencies of conversion of power into mechanical motion. Compressor 210 generates heat due to the increased resistance to movement of the compressor components by the air being compressed. Heat is also inherently generated due to adiabatic compression of the air by compressor 210. Thus, the continual pressurization of air produces heat in the enclosure. Additionally, power supply 180 may produce heat as power is supplied to compression system 200. Furthermore, users of the oxygen concentrator may operate the device in unconditioned environments (e.g., outdoors) at potentially higher ambient temperatures than indoors, thus the incoming air will already be in a heated state.

Heat produced inside oxygen concentrator 100 can be problematic. Lithium ion batteries are generally employed as internal power supplies for oxygen concentrators due to their long life and light weight. Lithium ion battery packs, however, are dangerous at elevated temperatures and safety controls are employed in oxygen concentrator 100 to shut down the system if dangerously high power supply temperatures are detected. Additionally, as the internal temperature of oxygen concentrator 100 increases, the amount of oxygen generated by the concentrator may decrease. This is due, in part, to the decreasing amount of oxygen in a given volume of air at higher temperatures. If the amount of produced oxygen drops below a predetermined amount, the oxygen concentrator 100 may automatically shut down.

Because of the compact nature of oxygen concentrators, dissipation of heat can be difficult. Solutions typically involve the use of one or more fans to create a flow of cooling air through the enclosure. Such solutions, however, require additional power from the power supply 180 and thus shorten the portable usage time of the oxygen concentrator. In an implementation, a passive cooling system may be used that takes advantage of the mechanical power produced by motor 220. Referring to FIGS. 1D and 1E, compression system 200 includes motor 220 having an external rotating armature (or external rotatable armature) 230. Specifically, armature 230 of motor 220 (e.g. a DC motor) is wrapped around the stationary field that is driving the armature. Since motor 220 is a large contributor of heat to the overall system it is helpful to transfer heat off the motor and sweep it out of the enclosure. With the external high speed rotation, the relative velocity of the major component of the motor and the air in which it exists is very high. The surface area of the armature is larger if externally mounted than if it is internally mounted. Since the rate of heat exchange is proportional to the surface area and the square of the velocity, using a larger surface area armature mounted externally increases the ability of heat to be dissipated from motor 220. The gain in cooling efficiency by mounting the armature externally, allows the elimination of one or more cooling fans, thus reducing the weight and power consumption while maintaining the interior of the oxygen concentrator within the appropriate temperature range. Additionally, the rotation of the externally mounted armature creates movement of air proximate to the motor to create additional cooling.

Moreover, an external rotatable armature may help the efficiency of the motor, allowing less heat to be generated. A motor having an external armature operates similar to the way a flywheel works in an internal combustion engine. When the motor is driving the compressor, the resistance to rotation is low at low pressures. When the pressure of the compressed air is higher, the resistance to rotation of the motor is higher. As a result, the motor does not maintain consistent ideal rotational stability, but instead surges and slows down depending on the pressure demands of the compressor. This tendency of the motor to surge and then slow down is inefficient and therefore generates heat. Use of an external armature adds greater angular momentum to the motor which helps to compensate for the variable resistance experienced by the motor. Since the motor does not have to work as hard, the heat produced by the motor may be reduced.

In an implementation, cooling efficiency may be further increased by coupling an air transfer device 240 to external rotatable armature 230. In an implementation, air transfer device 240 is coupled to the external armature 230 such that rotation of the external armature 230 causes the air transfer device 240 to create an air flow that passes over at least a portion of the motor. In an implementation, air transfer device 240 includes one or more fan blades coupled to the external armature 230. In an implementation, a plurality of fan blades may be arranged in an annular ring such that the air transfer device 240 acts as an impeller that is rotated by movement of the external rotatable armature 230. As depicted in FIGS. 1D and 1E, air transfer device 240 may be mounted to an outer surface of the external armature 230, in alignment with the motor 220. The mounting of the air transfer device 240 to the armature 230 allows air flow to be directed toward the main portion of the external rotatable armature 230, providing a cooling effect during use. In an implementation, the air transfer device 240 directs air flow such that a majority of the external rotatable armature 230 is in the air flow path.

Further, referring to FIGS. 1D and 1E, air pressurized by compressor 210 exits compressor 210 at compressor outlet 212. A compressor outlet conduit 250 is coupled to compressor outlet 212 to transfer the compressed air to canister system 300. As noted previously, compression of air causes an increase in the temperature of the air. This increase in temperature can be detrimental to the efficiency of the oxygen concentrator. In order to reduce the temperature of the pressurized air, compressor outlet conduit 250 is placed in the air flow path produced by air transfer device 240. At least a portion of compressor outlet conduit 250 may be positioned proximate to motor 220. Thus, air flow, created by air transfer device 240, may contact both motor 220 and compressor outlet conduit 250. In one implementation, a majority of compressor outlet conduit 250 is positioned proximate to motor 220. In an implementation, the compressor outlet conduit 250 is coiled around motor 220, as depicted in FIG. 1E.

In an implementation, the compressor outlet conduit 250 is composed of a heat exchange metal. Heat exchange metals include, but are not limited to, aluminum, carbon steel, stainless steel, titanium, copper, copper-nickel alloys or other alloys formed from combinations of these metals. Thus, compressor outlet conduit 250 can act as a heat exchanger to remove heat that is inherently caused by compression of the air. By removing heat from the compressed air, the number of molecules in a given volume at a given pressure is increased. As a result, the amount of oxygen enriched air that can be generated by each canister during each PSA cycle may be increased.

The heat dissipation mechanisms described herein are either passive or make use of elements required for the oxygen concentrator 100. Thus, for example, dissipation of heat may be increased without using systems that require additional power. By not requiring substantial additional power, the run-time of the internal power supply 180 may be increased and the size and weight of the oxygen concentrator may be minimized. Likewise, use of an additional box fan or cooling unit may be eliminated. Eliminating such additional features reduces the weight and power consumption of the oxygen concentrator.

As discussed above, adiabatic compression of air causes the air temperature to increase. During venting of a canister in canister system 300, the pressure of the exhaust gas being vented from the canisters decreases. The adiabatic decompression of the gas leaving the canister causes the temperature of the exhaust gas to drop as it is vented. In an implementation, the cooled exhaust gas 327 vented from canister system 300 is directed toward power supply 180 and toward compression system 200. In an implementation, base 315 of canister system 300 receives the exhaust gas from the canisters. The exhaust gas 327 is directed through base 315 toward outlet 325 of the base 315 and toward power supply 180. The exhaust gas, as noted, is cooled due to decompression of the gases and therefore passively provide cooling to the power supply 180. When the compression system 200 is operated, the air transfer device 240 will gather the cooled exhaust gas 327 and direct the exhaust gas 327 toward the motor 220 of compression system 200. Fan 172 may also assist in directing the exhaust gas 327 across compression system 200 and out of the housing 170. In this manner, additional cooling may be obtained without requiring substantial further power from the power supply 180.

Canister System

Oxygen concentrator 100 may include at least two canisters, each canister including a gas separation adsorbent. The canisters of oxygen concentrator 100 may be disposed formed from a molded housing. In an implementation, canister system 300 includes two housing components 310 and 510, as depicted in FIG. 1I. In various implementations, the housing components 310 and 510 of the oxygen concentrator 100 may form a two-part molded plastic frame that defines two canisters 302 and 304 and accumulator 106. The housing components 310 and 510 may be formed separately and then coupled together. In some implementations, housing components 310 and 510 may be injection molded or compression molded. Housing components 310 and 510 may be made from a thermoplastic polymer such as polycarbonate, methylene carbide, polystyrene, acrylonitrile butadiene styrene (ABS), polypropylene, polyethylene, or polyvinyl chloride. In another implementation, housing components 310 and 510 may be made of a thermoset plastic or metal (such as stainless steel or a lightweight aluminum alloy). Lightweight materials may be used to reduce the weight of the oxygen concentrator 100. In some implementations, the two housings 310 and 510 may be fastened together using screws or bolts. Alternatively, housing components 310 and 510 may be laser or solvent welded together.

As shown, valve seats 322, 324, 332, and 334 and conduits 330 and 346 may be integrated into the housing component 310 to reduce the number of sealed connections needed throughout the air flow of the oxygen concentrator 100.

Air pathways/tubing between different sections in housing components 310 and 510 may take the form of molded conduits. Conduits in the form of molded channels for air pathways may occupy multiple planes in housing components 310 and 510. For example, the molded air conduits may be formed at different depths and at different positions in housing components 310 and 510. In some implementations, a majority or substantially all of the conduits may be integrated into the housing components 310 and 510 to reduce potential leak points.

In some implementations, prior to coupling housing components 310 and 510 together, O-rings may be placed between various points of housing components 310 and 510 to ensure that the housing components are properly sealed. In some implementations, components may be integrated and/or coupled separately to housing components 310 and 510. For example, tubing, flow restrictors (e.g., press fit flow restrictors), oxygen sensors, gas separation adsorbents, check valves, plugs, processors, power supplies, etc. may be coupled to housing components 310 and 510 before and/or after the housing components are coupled together.

In some implementations, apertures 337 leading to the exterior of housing components 310 and 510 may be used to insert devices such as flow restrictors. Apertures may also be used for increased moldability. One or more of the apertures may be plugged after molding (e.g., with a plastic plug). In some implementations, flow restrictors may be inserted into passages prior to inserting plugs to seal the passages. Press fit flow restrictors may have diameters that may allow a friction fit between the press fit flow restrictors and their respective apertures. In some implementations, an adhesive may be added to the exterior of the press fit flow restrictors to hold the press fit flow restrictors in place once inserted. In some implementations, the plugs may have a friction fit with their respective tubes (or may have an adhesive applied to their outer surface). The press fit flow restrictors and/or other components may be inserted and pressed into their respective apertures using a narrow tip tool or rod (e.g., with a diameter less than the diameter of the respective aperture). In some implementations, the press fit flow restrictors may be inserted into their respective tubes until they abut a feature in the tube to halt their insertion. For example, the feature may include a reduction in radius. Other features are also contemplated (e.g., a bump in the side of the tubing, threads, etc.). In some implementations, press fit flow restrictors may be molded into the housing components (e.g., as narrow tube segments).

In some implementations, spring baffle 139 may be placed into respective canister receiving portions of housing components 310 and 510 with the spring side of the baffle 139 facing the exit of the canister. Spring baffle 139 may apply force to gas separation adsorbent in the canister while also assisting in preventing gas separation adsorbent from entering the exit apertures. Use of a spring baffle 139 may keep the gas separation adsorbent compact while also allowing for expansion (e.g., thermal expansion). Keeping the gas separation adsorbent compact may prevent the gas separation adsorbent from fragmenting during movement of the oxygen concentrator 100.

In some implementations, filter 129 may be placed into respective canister receiving portions of housing components 310 and 510 facing the inlet of the respective canisters. The filter 129 removes particles from the feed gas stream entering the canisters.

In some implementations, pressurized air from the compression system 200 may enter air inlet 306. Air inlet 306 is coupled to inlet conduit 330. Air enters housing component 310 through inlet 306 and travels through inlet conduit 330, and then to valve seats 322 and 324. FIG. 1J and FIG. 1K depict an end view of housing component 310. FIG. 1J depicts an end view of housing component 310 prior to fitting valves to housing component 310. FIG. 1K depicts an end view of housing component 310 with the valves fitted to the housing component 310. Valve seats 322 and 324 are configured to receive inlet valves 122 and 124 respectively. Inlet valve 122 is coupled to canister 302 and inlet valve 124 is coupled to canister 304. Inlet valves 122/124 are used to control the passage of air from inlet conduit 330 to the respective canisters.

In an implementation, pressurized air is fed into one of canisters 302 or 304 while the other canister is being vented. Valve seat 322 includes an opening 323 that passes through housing component 310 into canister 302. Similarly valve seat 324 includes an opening 375 that passes through housing component 310 into canister 304. Air from inlet conduit 330 passes through openings 323 or 375 if the respective valves 122 and 124 are de-actuated, and enters the respective canisters 302 and 304.

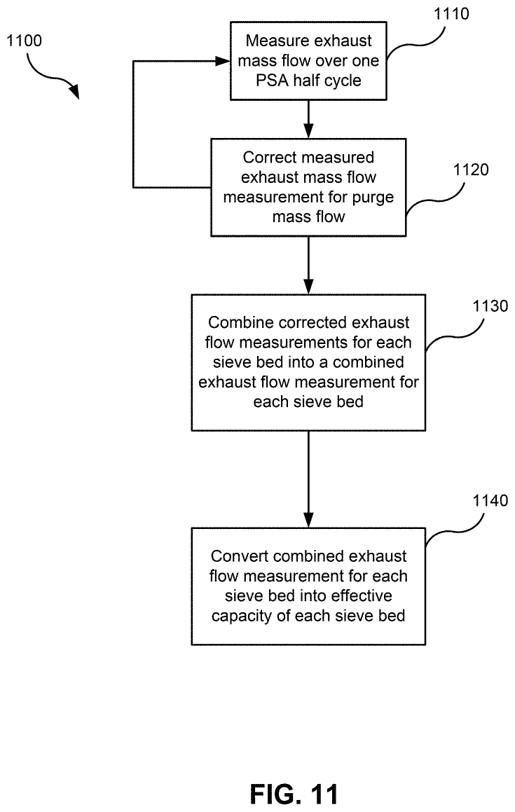

Check valves 142 and 144 (See FIG. 11) are coupled to canisters 302 and 304, respectively. Check valves 142 and 144 are one way valves that are passively operated by the pressure differentials that occur as the canisters are pressurized and vented. Oxygen enriched air produced in canisters 302 and 304 passes from the canisters into openings 542 and 544 of housing component 510. A passage (not shown) links openings 542 and 544 to conduits 342 and 344, respectively. Oxygen enriched air produced in canister 302 passes from the canister though opening 542 and into conduit 342 when the pressure in the canister is sufficient to open check valve 142. When check valve 142 is open, oxygen enriched air flows through conduit 342 toward the end of housing component 310. Similarly, oxygen enriched air produced in canister 304 passes from the canister through opening 544 and into conduit 344 when the pressure in the canister is sufficient to open check valve 144. When check valve 144 is open, oxygen enriched air flows through conduit 344 toward the end of housing component 310.

Oxygen enriched air from either canister travels through conduit 342 or 344 and enters conduit 346 formed in housing component 310. Conduit 346 includes openings that couple the conduit to conduit 342, conduit 344 and accumulator 106. Thus, oxygen enriched air, produced in canister 302 or 304, travels to conduit 346 and passes into accumulator 106. As illustrated in FIG. 1B, gas pressure within the accumulator 106 may be measured by a sensor, such as with an accumulator pressure sensor 107. (See also FIG. 1F.) Thus, the accumulator pressure sensor 107 generates a signal representing the pressure of the accumulated oxygen enriched air. An example of a suitable pressure transducer is a sensor from the HONEYWELL ASDX series. An alternative suitable pressure transducer is a sensor from the NPA Series from GENERAL ELECTRIC. In some implementations, the pressure sensor 107 may alternatively measure pressure of the gas outside of the accumulator 106, such as in an output path between the accumulator 106 and a valve (e.g., supply valve 160) that controls the release of the oxygen enriched air for delivery to a user in a bolus.

Canister 302 is vented by actuating inlet valve 122, releasing the exhaust gas from canister 302 into the volume defined by the end of housing component 310. Foam material may cover the end of housing component 310 to reduce the sound made by release of gases from the canisters. Similarly, canister 304 is vented by actuating inlet valve 124, releasing the exhaust gas from canister 304 into the volume defined by the end of housing component 310.

Two conduits are formed in housing component 510 for use in transferring oxygen enriched air between canisters. As shown in FIG. 1L, conduit 530 couples canister 302 to canister 304. Conduit 530 is coupled to valve seat 554 which receives valve 154, as shown in FIG. 1M. Flow restrictors 153 and 155 (not shown) are disposed in conduit 530, between canister 302 and 304, to restrict flow of oxygen enriched air during purging. The valve 154 works with flow restrictors 153 and 155 to optimize the purge flow balance between the two canisters. Conduit 532 also couples canister 302 to 304. Conduit 532 is coupled to valve seat 552 which receives valve 152, as shown in FIG. 1M.

Oxygen enriched air in accumulator 106 passes through supply valve 160 as described below. An opening (not shown) in housing component 510 couples accumulator 106 to supply valve 160.

Outlet System

An outlet system, coupled to one or more of the canisters, includes one or more conduits for providing oxygen enriched air to a user. In an implementation, oxygen enriched air produced in either of canisters 302 and 304 is collected in accumulator 106 through check valves 142 and 144, respectively, as depicted schematically in FIG. 1B. The oxygen enriched air leaving the canisters may be collected in an oxygen accumulator 106 prior to being provided to a user. In some implementations, a conduit such as a tube may be coupled to the accumulator 106 to provide the oxygen enriched air to the user. Oxygen enriched air may be provided to the user through an airway delivery device that transfers the oxygen enriched air to the user's mouth and/or nose. In an implementation, the airway delivery device may include a tube that directs the oxygen toward a user's nose and/or mouth that may not be directly coupled to the user's nose.

Turning to FIG. 1F, a schematic diagram of an implementation of an outlet system 150 for an oxygen concentrator is shown. A supply valve 160 (labelled “F” and sometimes referred to as the “F-valve”) may be coupled to a conduit to control the release of the oxygen enriched air from accumulator 106 to the user. Supply valve 160 is actuated by controller 400 to control the delivery of oxygen enriched air to a user. In an implementation, supply valve 160 is an electromagnetically actuated plunger valve. Actuation of supply valve 160 is not timed or synchronized to the pressure swing adsorption process. Instead, actuation is synchronized to the user's breathing as described below. In some implementations, supply valve 160 may have continuously-valued actuation to establish a clinically effective amplitude profile for providing oxygen enriched air.

Oxygen enriched air in accumulator 106 passes through supply valve 160 into oxygen sensor 165 as depicted in FIG. 1F. In an implementation, oxygen sensor 165 may include one or more devices configured to estimate an oxygen concentration of gas passing through the oxygen sensor 165. Oxygen enriched air is released from accumulator 106 by supply valve 160, and then is bled through a small orifice flow restrictor 175 to oxygen sensor 165 and then to particulate filter 187. Flow restrictor 175 may be a 0.25 D flow restrictor. Other flow restrictor types and sizes may be used. In some implementations, the diameter of the air pathway in the housing may be restricted to create restricted gas flow. Particulate filter 187 may be used to filter bacteria, dust, granule particles, etc., prior to delivery of the oxygen enriched air to the user. The oxygen enriched air passes through filter 187 to connector 190 which sends the oxygen enriched air to the user via delivery conduit 192 and to pressure sensor 194.

Oxygen sensor 165 is a device configured to measure oxygen concentration in a gas. Examples of oxygen sensors include, but are not limited to, ultrasonic oxygen sensors, electrical oxygen sensors, chemical oxygen sensors, and optical oxygen sensors. In one implementation, oxygen sensor 165 is a chemical oxygen sensor. Implementations of chemical oxygen sensors are discussed in more detail in U.S. Provisional Patent Application No. 62/941,763, filed on 28 Nov. 2019, the entire disclosure of which is incorporated herein by reference.

Particulate filter 187 removes bacteria, dust, granule particles, etc prior to providing the oxygen enriched air to the user. The filtered oxygen enriched air passes to connector 190. Connector 190 may be a “Y” connector coupling the outlet of filter 187 to pressure sensor 194 and delivery conduit 192. Pressure sensor 194 may be used to monitor the pressure of the gas passing through delivery conduit 192 to the user. In some implementations, pressure sensor 194 is configured to generate a signal that is proportional to the amount of positive or negative pressure applied to a sensing surface. In an implementation, controller 400 may control actuation of supply valve 160 based on information provided by the pressure sensor 194. Changes in pressure, sensed by pressure sensor 194, may be used to determine a breathing rate of a user, as well as the onset of inhalation as described below. In POD mode, controller 400 may control actuation of supply valve 160 based on the breathing rate and/or onset of inhalation of the user.

Oxygen enriched air may be provided to a user through delivery conduit 192. In an implementation, delivery conduit 192 may be a silicone tube. Delivery conduit 192 may be coupled to a user using an airway delivery device 196, as depicted in FIGS. 1G and 1H. An airway delivery device 196 may be any device capable of providing the oxygen enriched air to nasal cavities or oral cavities. Examples of airway delivery devices include, but are not limited to: nasal masks, nasal pillows, nasal prongs, nasal cannulas, and mouthpieces. A nasal cannula airway delivery device 196 is depicted in FIG. 1G. Nasal cannula airway delivery device 196 is positioned proximate to a user's airway (e.g., proximate to the user's mouth and or nose) to allow delivery of the oxygen enriched air to the user while allowing the user to breathe air from the surroundings.

In an alternate implementation, a mouthpiece may be used to provide oxygen enriched air to the user. As shown in FIG. 1H, a mouthpiece 198 may be coupled to oxygen concentrator 100. Mouthpiece 198 may be the only device used to provide oxygen enriched air to the user, or a mouthpiece may be used in combination with a nasal delivery device (e.g., a nasal cannula). As depicted in FIG. 1H, oxygen enriched air may be provided to a user through both nasal cannula airway delivery device 196 and mouthpiece 198.

Mouthpiece 198 is removably positionable in a user's mouth. In one implementation, mouthpiece 198 is removably couplable to one or more teeth in a user's mouth. During use, oxygen enriched air is directed into the user's mouth via the mouthpiece. Mouthpiece 198 may be a night guard mouthpiece which is molded to conform to the user's teeth. Alternatively, mouthpiece may be a mandibular repositioning device. In an implementation, at least a majority of the mouthpiece is positioned in a user's mouth during use.

During use, oxygen enriched air may be released to mouthpiece 198 when a change in pressure is detected proximate to the mouthpiece. In one implementation, mouthpiece 198 may be coupled to a pressure sensor 194. When a user inhales air through the user's mouth, pressure sensor 194 may detect a change in pressure proximate to the mouthpiece 198. Changes in pressure, sensed by pressure sensor 194, may be used to determine a breathing rate of a user, as well as the onset of inhalation as described below. In POD mode, controller 400 of oxygen concentrator 100 may control actuation of supply valve 160 based on the breathing rate and/or onset of inhalation of the user.

During typical breathing of an individual, inhalation may occur through the nose, through the mouth or through both the nose and the mouth. Furthermore, breathing may change from one passageway to another depending on a variety of factors. For example, during more active activities, a user may switch from breathing through their nose to breathing through their mouth, or breathing through their mouth and nose. A system that relies on a single mode of delivery (either nasal or oral), may not function properly if breathing through the monitored pathway is stopped. For example, if a nasal cannula is used to provide oxygen enriched air to the user, an inhalation sensor (e.g., a pressure sensor) may be coupled to the nasal cannula to determine the onset of inhalation. If the user stops breathing through their nose, and switches to breathing through their mouth, the oxygen concentrator 100 may not know when to provide the oxygen enriched air since there is no pressure change in the nasal cannula. Under such circumstances, oxygen concentrator 100 may increase the flow rate and/or increase the frequency of providing oxygen enriched air until the inhalation sensor detects an inhalation by the user. If the user switches between breathing modes often, the default mode of providing oxygen enriched air may cause the oxygen concentrator 100 to work harder, limiting the portable usage time of the system.

In an implementation, mouthpiece 198 is used in combination with nasal cannula airway delivery device 196 to provide oxygen enriched air to a user, as depicted in FIG. 1H. Both mouthpiece 198 and nasal cannula airway delivery device 196 are coupled to an inhalation sensor. In one implementation, mouthpiece 198 and nasal cannula airway delivery device 196 are coupled to the same inhalation sensor. In an alternate implementation, mouthpiece 198 and nasal cannula airway delivery device 196 are coupled to different inhalation sensors. In either implementation, the inhalation sensor(s) may detect the onset of inhalation from either the mouth or the nose. Oxygen concentrator 100 may be configured to provide oxygen enriched air to the delivery device (i.e. mouthpiece 198 or nasal cannula airway delivery device 196) proximate to which the onset of inhalation was detected. Alternatively, oxygen enriched air may be provided to both mouthpiece 198 and nasal cannula airway delivery device 196 if onset of inhalation is detected proximate either delivery device. The use of a dual delivery system, such as depicted in FIG. 1H, may be particularly useful for users when they are sleeping and may switch between nose breathing and mouth breathing without conscious effort.

Controller System

Operation of oxygen concentrator 100 may be performed automatically using an internal controller 400 coupled to various components of the oxygen concentrator 100, as described herein. The controller 400 may be implemented by one or more hardware components (e.g., hardware controller(s)) and may be implemented with one or more programming logic or software controllers that are programming logic modules of a hardware controller. Thus, controller 400 may include one or more processors 410 and internal memory 420, as depicted in FIG. 1B. Methods used to operate and monitor oxygen concentrator 100 may be implemented by program instructions stored in internal memory 420 or an external memory medium coupled to controller 400, and executed by one or more processors 410. A memory medium may include any of various types of memory devices or storage devices. The term “memory medium” is intended to include an installation medium, e.g., a Compact Disc Read Only Memory (CD-ROM), floppy disks, or tape device; a computer system memory or random access memory such as Dynamic Random Access Memory (DRAM), Double Data Rate Random Access Memory (DDR RAM), Static Random Access Memory (SRAM), Extended Data Out Random Access Memory (EDO RAM), Random Access Memory (RAM), etc.; or a non-volatile memory such as a magnetic medium, e.g., a hard drive, or optical storage. The memory medium may comprise other types of memory as well, or combinations thereof. In addition, the memory medium may be located proximate to the controller 400 by which the programs are executed, or may be located in an external computing device that connects to the controller 400 over a network, as described below. In the latter instance, the external computing device may provide program instructions to the controller 400 for execution. The term “memory medium” may include two or more memory media that may reside in different locations, e.g., in different computing devices that are connected over a network.

In some implementations, controller 400 includes processor 410 that includes, for example, one or more field programmable gate arrays (FPGAs), microcontrollers, etc. included on a circuit board disposed in oxygen concentrator 100. Processor 410 is configured to execute programming instructions stored in memory 420. In some implementations, programming instructions may be built into processor 410 such that a memory external to the processor 410 may not be separately accessed (i.e., the memory 420 may be internal to the processor 410).

Processor 410 may be coupled to various components of oxygen concentrator 100, including, but not limited to compression system 200, one or more of the valves used to control fluid flow through the system (e.g., valves 122, 124, 152, 154, 160), oxygen sensor 165, pressure sensor 194, temperature sensors (not shown), fan 172, and any other component that may be electrically controlled or sensed. In some implementations, a separate processor (and/or memory) may be coupled to one or more of the components.

Controller 400 is configured (e.g. programmed by program instructions) to operate oxygen concentrator 100 and is further configured to monitor the oxygen concentrator 100 such as for malfunction states or other process information. For example, in one implementation, controller 400 is programmed to trigger an alarm if the system is operating and no breathing is detected by the user for a predetermined amount of time. For example, if controller 400 does not detect a breath for a period of 75 seconds, an alarm LED may be lit and/or an audible alarm may be sounded. If the user has truly stopped breathing, for example, during a sleep apnea episode, the alarm may be sufficient to awaken the user, causing the user to resume breathing. The action of breathing may be sufficient for controller 400 to reset this alarm function. Alternatively, if the system is accidentally left on when delivery conduit 192 is removed from the user, the alarm may serve as a reminder for the user to turn oxygen concentrator 100 off.

Controller 400 is further coupled to oxygen sensor 165, and may be programmed for continuous or periodic monitoring of the oxygen concentration of the oxygen enriched air passing through oxygen sensor 165. A minimum oxygen concentration threshold may be programmed into controller 400, such that the controller lights an LED visual alarm and/or an audible alarm to warn the user of the low concentration of oxygen.

Controller 400 is also coupled to internal power supply 180 and may be configured to monitor the level of charge of the internal power supply. A minimum voltage and/or current threshold may be programmed into controller 400, such that the controller lights an LED visual alarm and/or an audible alarm to warn the user of low power condition. The alarms may be activated intermittently and at an increasing frequency as the internal power supply approaches zero usable charge.

FIG. 1O illustrates one implementation of a connected oxygen therapy system 450 including the POC 100. Controller 400 of the POC 100 includes the transceiver 430 configured to allow the controller 400 to communicate, using a wireless communication protocol such as the Global System for Mobile Telephony (GSM) or other protocol (e.g., WiFi), with a remote computing device such as a cloud-based server 460 such as over a network 470. The network 470 may be a wide-area network such as the Internet, or a local-area network such as an Ethernet. The controller 400 may also include a short range wireless module in the transceiver 430 configured to enable the controller 400 to communicate, using a short range wireless communication protocol such as Bluetooth™, with a portable computing device 480 such as a smartphone. The portable computing device, e.g. smartphone, 480 may be associated with a user 500 of the POC 100.

The server 460 may also be in wireless communication with the portable computing device 480 using a wireless communication protocol such as GSM. A processor of the smartphone 480 may execute a program 482 known as an “app” to control the interaction of the smartphone 480 with the user 500, the POC 100, and/or the server 460. The server 460 may have access to a database 466 that stores operational data about the POC 100 and user 500.

The server 460 includes an analysis engine 462 that may execute methods of operating and monitoring the POC 100 as further described below. The server 460 may also be in communication via the network 470 with other devices such as a personal computing device (e.g. a workstation) 464 via a wired or wireless connection. A processor of the personal computing device 464 may execute a “client” program to control the interaction of the personal computing device 464 with the server 460. One example of a client program is a browser.

In a further implementation, the server 460 may be configured to host a portal system. The portal system may receive, from the portable computing device 480 or directly from the POC 100, data relating to the operation of the POC 100. As described above, the personal computing device 464 may execute a client program such as a browser to allow a user of the personal computing device 464 (such as a representative of a home medical equipment provider) to access the operational data of the POC 100, and other POCs in the connected oxygen therapy system 450, via the portal system hosted by the server 460. In this fashion, such a portal system may be utilised by an HME to manage a population of users of POC devices, e.g. the POC 100, in the connected oxygen therapy system 450. The portal system may provide actionable insights into user or device condition for the population of POC devices and their users based on the operational data received by the portal system. Such insights may be based on rules that are applied to the operational data.

Further functions that may be implemented with or by the controller 400 are described below. For example, the controller of the POC may implement compressor control to regulate pressure in the system. Thus, the POC may be equipped with a pressure sensor such as the pressure sensor 107 in the accumulator 106 downstream of the canisters 302 and 304. The controller 400 in the POC can control adjusting of the speed of the compressor 210 using signals from the pressure sensor as well as a motor speed sensor such as in one or more modes. In this regard, the controller may implement dual control modes, designated a coarse pressure regulation mode and a fine pressure regulation mode. The coarse pressure regulation mode may be implemented for changing between the different flow rate settings of the POC and for starting/initial activation. The fine pressure regulation mode may then take over upon completion of each operation of the coarse pressure regulation mode.

In the coarse pressure regulation mode, the motor speed is set/controlled to ramp up or down depending the prior state of operations. During the ramping, the controller uses the measurements from the pressure sensor 107 to generate an estimated pressure upstream of the pressure sensor 107, in the canisters. In some implementations, the estimated pressure is used in a test to interrupt the ramp, e.g. when the estimated pressure reaches a predetermined target pressure value that is associated with the selected flow rate setting of the POC. Table 4 contains example target pressure values associated with each of six flow rate settings and flow rates listed in Table 1 according to one implementation of the present technology.

TABLE 4
Example target pressure values at each of the six
flow rate settings in Table 1.

| Flow rate setting | Flow rate (LPM) | Target pressure (kPa) |
|---|---|---|
| 1 | 0.2 | 45 |
| 2 | 0.4 | 60 |
| 3 | 0.6 | 75 |
| 4 | 0.8 | 95 |
| 5 | 1.0 | 115 |
| 6 | 1.1 | 125 |

Other target pressure values may also be used, depending on the type of gas separation adsorbent disposed in the canisters.

The pressure estimate may be calculated by performing a regression (e.g., linear) using data from the pressure sensor whereby the controller determines regression parameters (e.g., slope and intercept parameters of a line) from the sensor signal samples. The pressure estimate is calculated with the regression parameters and a known system response delay.

In the fine pressure regulation mode, the motor speed is controlled to maintain the pressure of the system at the target pressure value using the signal from the pressure sensor 107. Upon completion of the coarse pressure regulation mode, the motor speed ramping is interrupted and the motor speed set point is initialised to the current motor speed. Any further changes to the motor speed set point are implemented by a fine pressure controller such as a PID (proportional, integral, derivative) controller. During the fine pressure regulation mode, the target pressure is compared with a qualified pressure estimate to generate a first error signal that is applied to the fine pressure controller to produce a speed adjustment. By adding the speed adjustment to the current motor speed set point, the speed set point for the motor may be adjusted. The speed set point is used for control of the motor speed using a motor control circuit, e.g. as described above in relation to FIG. 3.

The qualified pressure estimate for the fine pressure controller is computed using regression. In this regard, samples from the pressure signal may be applied to a best fit algorithm (e.g., linear regression) to determine regression parameters (e.g., slope and intercept of a line) of the data from the pressure signal during an adsorption phase of the PSA cycle. If the slope is positive, these parameters (slope and intercept rather than pressure samples from the pressure sensor) may then be applied with the particular time of the given adsorption phase of the PSA cycle to determine a peak value by extrapolating the regression line obtained from the linear regression. If the slope is negative, the intercept parameter may be taken as the peak value. The peak values from the regression information may be then applied to a running average buffer that maintains an average of the most recent peak values (e.g., six or more). The average peak value may then serve as the qualified pressure estimate for the fine pressure controller. Implementations of such processes are discussed in more detail in Patent Cooperation Treaty (PCT) Application No. PCT/AU2020/051015, filed on 24 Sep. 2020, the entire disclosure of which is incorporated herein by reference.

Control Panel

Control panel 600 serves as an interface between a user and controller 400 to allow the user to initiate predetermined operation modes of the oxygen concentrator 100 and to monitor the status of the system. FIG. 1N depicts an implementation of control panel 600. Charging input port 605, for charging the internal power supply 180, may be disposed in control panel 600.

In some implementations, control panel 600 may include buttons to activate various operation modes for the oxygen concentrator 100. For example, control panel may include power button 610, flow rate setting buttons 620 to 626, active mode button 630, sleep mode button 635, altitude button 640, and a battery check button 650. In some implementations, one or more of the buttons may have a corresponding LED that may illuminate when the corresponding button is pressed, and may be extinguished when the button is pressed again. Power button 610 may power the system on or off. If the power button 610 is activated to turn the system off, controller 400 may initiate a shutdown sequence to place the system in a shutdown state (e.g., a state in which both canisters are pressurized).

Flow rate setting buttons 620, 622, 624, and 626 allow a flow rate of oxygen enriched air to be selected (e.g., 0.2 LPM by button 620, 0.4 LPM by button 622, 0.6 LPM by button 624, and 0.8 LPM by button 626). In other implementations, the number of flow rate settings may be increased or decreased. After a flow rate setting is selected, oxygen concentrator 100 will then control operations to achieve production of the oxygen enriched air according to the selected flow rate setting.

Altitude button 640 may be activated when a user is going to be in a location at a higher elevation than the oxygen concentrator 100 is regularly used by the user.

Battery check button 650 initiates a battery check routine in the oxygen concentrator 100 which results in a relative battery power remaining LED 655 being illuminated on control panel 600.

Methods of Operating the POC

The methods of operating and monitoring the POC 100 described below may be executed by the one or more processors, such as the one or more processors 410 of the controller 400, configured by program instructions, such as including, as previously described, the one or more functions and/or associated data corresponding thereto, stored in a memory such as the memory 420 of the POC 100. Alternatively, some or all of the steps of the described methods may be similarly executed by one or more processors of an external computing device, such as the server 460, forming part of the connected oxygen therapy system 450, as described above. In this latter implementation, the processors 410 may be configured by program instructions stored in the memory 420 of the POC 100 to transmit to the external computing device the measurements and parameters necessary for the performance of those steps that are to be carried out at the external computing device.

Triggering

The main use of an oxygen concentrator 100 is to provide supplemental oxygen to a user. One or more flow rate settings may be selected on a control panel 600 of the oxygen concentrator 100, which then will control operations to achieve production of the oxygen enriched air according to the selected flow rate setting. In some implementations, a plurality of flow rate settings may be implemented (e.g., five flow rate settings). As described in more detail herein, the controller 400 may implement a POD (pulsed oxygen delivery) or demand mode of operation.

In order to maximise the effect of the delivered oxygen enriched air, controller 400 may be programmed to synchronise the release of each bolus of the oxygen enriched air with the user's inhalations. Releasing a bolus of oxygen enriched air to the user as the user inhales may reduce wastage of oxygen by not releasing oxygen, for example, when the user is exhaling.

Oxygen enriched air produced by oxygen concentrator 100 is stored in an oxygen accumulator 106 and, in a POD mode of operation, released to the user as the user inhales. In order to minimize the wastage of oxygen, the oxygen enriched air may be released as a bolus soon after the onset of a user's inhalation is detected. For example, the bolus of oxygen enriched air may be released in the first few milliseconds of a user's inhalation.

In an implementation, pressure sensor 194 may be used as an inhalation sensor to detect the onset of inhalation by the user (a process referred to as “triggering”). In use, delivery conduit 192 for providing oxygen enriched air is coupled to the user's nose and/or mouth through the nasal cannula airway delivery device 196 and/or mouthpiece 198. The pressure in delivery conduit 192 is therefore representative of the user's airway pressure and hence indicative of user respiration. At the onset of inhalation, the user begins to draw air into their body through the nose and/or mouth. As the air is drawn in, a negative pressure is generated at the end of delivery conduit 192, due, in part, to the venturi action of the air being drawn across the end of delivery conduit 192. Controller 400 analyses the pressure signal from the pressure sensor 194 to detect the onset of inhalation. Upon detection of the onset of inhalation, supply valve 160 is opened to release a bolus of oxygen enriched air from the accumulator 106.

By measuring the intervals between adjacent onsets of inhalation, the user's breathing rate may be estimated. By measuring the intervals between onsets of inhalation and the subsequent onsets of exhalation, the user's inspiratory time may be estimated.

In other implementations, the pressure sensor 194 may be located in a sensing conduit that is in pneumatic communication with the user's airway, but separate from the delivery conduit 192. In such implementations the pressure signal from the pressure sensor 194 is therefore also representative of the user's airway pressure.

In some implementations, the sensitivity of the pressure sensor 194 may be affected by the physical distance of the pressure sensor 194 from the user, especially if the pressure sensor 194 is located in oxygen concentrator 100 and the pressure difference is detected through delivery conduit 192 coupling the oxygen concentrator 100 to the user. In some implementations, the pressure sensor 194 may be placed in the airway delivery device 196 used to provide the oxygen enriched air to the user. A signal from the pressure sensor 194 may be provided to controller 400 in the oxygen concentrator 100 electronically via a wire or through telemetry such as through Bluetooth™ or other wireless technology.

The sensitivity of the triggering process is governed by a trigger threshold. The signal from the pressure sensor 194 is compared with the trigger threshold to determine whether a significant drop in pressure has taken place, thereby indicating onset of inhalation. However, it is difficult to set a static trigger threshold that remains accurate under all conditions such that most inhalations are detected while largely avoiding “false triggering”.

A user may have a low breathing rate or depth if relatively inactive (e.g., asleep, sitting, etc.), or a high breathing rate or depth if relatively active (e.g., walking, exercising, etc.). In some implementations, the POC 100 defaults to active mode, and sleep mode may be entered automatically by comparing the estimated breathing rate or depth to a threshold. Additionally, or alternatively, the user may manually request the POC 100 to enter active mode or sleep mode by pressing button 630 for active mode or button 635 for sleep mode respectively.

In some such implementations, the trigger threshold is set to give the triggering process a higher sensitivity when the POC 100 is in sleep mode (e.g. as entered automatically or as requested by the user via the sleep mode button 635) compared to when the POC 100 is in active mode (e.g. as entered automatically or as requested by the user via the active mode button 630).

In some such implementations, if the POC 100 is in active mode and an onset of inhalation has not been detected for a predetermined interval, e.g. 8 seconds, the POC 100 enters sleep mode, which increases the trigger sensitivity as described above.

Bolus Size Regulation

The amount of oxygen enriched air provided by oxygen concentrator 100 is controlled, in part, by supply valve 160. In POD mode, the controller 400 of the POC may be configured to implement control of supply valve 160 to regulate the size (e.g. volume) of each released bolus of oxygen enriched air according to the selected flow rate setting. Bolus size is typically regulated by actuating the supply valve 160 for a fixed time, where the fixed time starts with the opening of the supply valve 160 to permit release of the bolus and ends when the supply valve 160 is closed thereby to stop releasing the bolus. The fixed time is calibrated to be associated with a desired or target bolus size based on the selected flow rate setting. However, such a fixed-time process does not always achieve the target bolus size. For example, system characteristics such as compressor variability as well as the adsorption process (e.g., the PSA cycle, sieve bed condition, air filter condition etc.) can cause the delivered bolus size to depart from the target bolus size.

In some implementations, bolus release control may be implemented with a dynamic timing parameter (e.g., a timing threshold), rather than a fixed one, that may take into account system conditions during release of the bolus so that the bolus control may more accurately achieve the desired bolus size. Thus, the timing threshold for ceasing the bolus release may change during the release of the bolus depending on system conditions (e.g., accumulator pressure). For example, the release of a bolus may be implemented by the controller 400 according to a function of a value of a measured pressure signal from a pressure sensor, such as the accumulator pressure sensor 107, and a target duration of supply valve opening for the bolus. The controller may dynamically determine, calculate (or recalculate) the target duration during the release of the bolus, such as with the function. Moreover, the function may include one or more parameters (e.g., empirical constants) of a pressure-size-time modelling surface that is derived from a calibration process utilizing measured pressure, size, and supply valve opening times. Parameters for such a modelling surface may optionally be derived by a fitting process such as regression utilizing measurements obtained during the calibration process. The calibration may be conducted for each POC 100 individually, or for a single POC that is representative of multiple POCs with similar outlet pneumatic characteristics.

The modelling surface maps accumulator pressure and supply valve opening times to bolus size, such as for one or more flow settings of the portable oxygen concentrator 100, and may include one or more parameters. With measured values of bolus size delivered by the POC, a modelling surface may be derived from or fitted to the data, such as by regression or best fit analysis, to derive the parameters of the modelling surface.

The modelled surface may be bilinear or of another suitable form. In one example utilizing pressure and supply valve opening times for a range of bolus sizes, a suitable functional form for the modelling surface may be as follows:

BolusSize=a*P+b*P*Fₜᵢₘₑ+c*Fₜᵢₘₑ+d (1)

where:
BolusSize is a measured size for the bolus such as a volume in milliliters, or number of moles of gas;
P is a value of measured pressure, such as an average pressure during bolus release, or a pressure measure at the time of initial bolus release;
Fₜᵢₘₑ is a duration or period of time that the supply valve is open during release of the bolus; and
a, b, c, and d are parameters derived from a fitting process applied to the calibration process measurements.

In some implementations, a determined set of these parameters may be associated with each flow rate setting of the oxygen concentrator. Thus, when Equation (1) is applied by a controller in a POD mode, the controller may access a particular set of parameters that is associated with a current flow rate setting of the POC. Thus, the controller may have a plurality of discrete sets of parameters for the modelling surface that are respectively associated with different flow rate settings of the oxygen concentrator. In other implementations, a single set of parameters may be derived and applied for all flow rate settings. Implementations of such processes are discussed in more detail in U.S. Provisional Application No. 62/932,125, filed on 7 Nov. 2019, the entire disclosure of which is incorporated herein by reference.

Estimation of Effective Capacity

As described above, during the pressurisation phase of a PSA cycle for either canister (302 or 304), feed gas is fed in to the canister to increase the pressure of the sieve bed. After the pressurisation phase, the adsorption phase commences during which the sieve bed pressure stabilises or rises more slowly than during the pressurization phase. After the adsorption phase, the sieve bed is partially depressurized by actuation of the E-valve 152 (equalization (1) and (2) phases). The equalization phases are followed by the desorb/vent phase in which the sieve bed vents its exhaust gas, and the purge phase in which further nitrogen is desorbed from the adsorbent and purged from the sieve bed by a flow of oxygen enriched air from the other canister.

As the adsorbent in a sieve bed becomes degraded, less nitrogen is adsorbed by the adsorbent during the pressurisation and adsorption phases, and therefore more of the nitrogen is available to increase the pressure inside the sieve bed. The sieve bed pressure therefore increases more rapidly for given mass flow rate of feed gas. The sieve bed pressure increase rate and rise time during the pressurisation phase (before the check valve 142 or 144 opens) are thus indicative of the effective adsorption capacity of the adsorbent material in the sieve bed, assuming no change in ambient conditions or input mass flow rate. Likewise, as the adsorbent becomes degraded, and less nitrogen is adsorbed, less nitrogen is desorbed by a depressurising sieve bed. The amount of exhaust gas during the desorb/vent and purge phases is thus also indicative of the effective capacity.

There are typically two void spaces within a packed sieve bed. One is the void space between particles, which is the volume of the canister not occupied by the solid matter of the particles. This void space is called bed void. The other void space is within each particle as the adsorbent used in portable oxygen concentrator is porous. This void space is called particle void. The combination of these two void spaces compose the total void in a packed bed. The void volumes (bed void and particle void, which summed together may be considered the total void) are usually stated as a fraction, E.

Bed void fraction εₜ, is the ratio of bed void volume to canister volume. The bed void fraction εₜ, can be calculated as

$${ɛ}_{b} = \frac{{ρ}_{bulk}}{{ρ}_{p a r t t c l e}} \qquad ( 2 )$$

where ρ_{bulk} is the “bulk” density of the adsorbent in the canister (mass per unit volume) and ρ_{particle} is the “matter” density of the individual adsorbent particles (mass per unit volume, with the same units as ρ_{bulk}). The bulk density ρ_{bulk} is the ratio of the mass of adsorbent particles in a canister (a known canister parameter) to the volume V of the canister, typically 500 to 800 milligrams per cubic centimeter. The matter density ρ_{particle} is a quantity specified by the manufacturer of the adsorbent material, typically 900 to 1500 milligrams per cubic centimeter.

If ρ_{particle} is not known or not supplied by the manufacturer, then ε_{b} can be calculated from sieve bed pressure drop through the Ergun equation, known by those skilled in the art (Ergun, 1952).

Particle void fraction or εₚ is the ratio of particle void volume to particle volume. This value may be provided by the material manufacturer. If this value is not available through the material manufacturer, it can be computed through gas pycnometer data. A gas pycnometer can provide material skeletal density ρₛₖₑₗₑₜₐₗ which is mass to volume of the solid component in a particle (taking out all the void and empty spaces). Using this data and ρ_{particle}, the particle void fraction εₚ can be calculated as

$${ɛ}_{p} = 1 - \frac{{ρ}_{particle}}{{ρ}_{s k e l e t a l}} \qquad ( 3 )$$

Based on both εₚ and ε_{b}, a total void fraction εₜₒₜₐₗ can be calculated as

εₜₒₜₐₗ=ε_{b}+εₚ−(ε_{b}×εₚ) (4)

When feed gas molecules are fed into the sieve bed during the pressurisation phase, they either enter the void (in which case they increase the pressure in the canister) or are adsorbed by the adsorbent (in which case they do not increase the pressure in the canister). The total number N of moles of feed gas fed into the sieve bed over the pressurisation phase is made up of the moo moles that entered the void and n_{ads} moles that were adsorbed by the adsorbent:

N=n_{void}+n_{ads} (5)

If the sieve bed pressure increases by ΔP during the pressurisation phase, then the number n_{void} of moles of gas that have increased the pressure, i.e. entered the void, may be computed from the ideal gas equation as follows:

$${n}_{void} = \frac{Δ P {V}_{void}}{R T} \qquad ( 6 )$$

where R is the universal gas constant (approximately equal to 8.31 in SI units), T is the temperature of the feed gas, and V_{void} is the void volume, i.e. the total void fraction εₜₒₜₐₗ of equation (4) times the volume V of the space being pressurised.

Equation (6) may be written in differential form as follows:

$$\frac{d {n}_{void}}{d t} = {P}^{′} \frac{R T}{{ɛ}_{total} V} \qquad ( 7 )$$

In other words, the rate of molecules entering the void is proportional to the rate P′ of pressure increase with respect to time at the start of the pressurisation phase, assuming all the other terms remain constant.

The rate of molecules entering the void is also proportional to the rate at which they are fed in to the canister from the compressor, regardless of the state of the adsorbent. This feed mass flow rate may vary with time. To remove this as a source of variation, the compressor mass flow rate Q (in moles per second) may be normalised out of equation (7) to obtain a “void flow fraction” x representing the fraction of molecules entering the void per unit time:

$$x = \frac{1}{Q} \frac{d {n}_{void}}{d t} = \frac{{P}^{′}}{Q} \frac{R T}{{ɛ}_{total} V} \qquad ( 8 )$$

The void flow fraction x is a measure of the degradation of the sieve bed, in that when the sieve bed is completely saturated with water, the void flow fraction x will be equal to one, as no input gas molecules will be adsorbed, while the void flow fraction x will be lower than one for a fresh sieve bed. The effective capacity C of the sieve bed is inversely related to the void flow fraction x, in that as the value of the void flow fraction x increases, the effective capacity decreases over the sieve bed life.

Calculating the void flow fraction x requires values for R, εₜₒₜₐₗ, V, and T. However, since R, εₜₒₜₐₗ and V are constant, the rate P′ of sieve bed pressure increase at the start of the pressurisation phase may be used as a proxy for the void flow fraction x, on the assumption that the temperature T and the mass flow rate Q can be either held constant for all measurements over the life of the sieve bed, or that any variations therein between measurements can be compensated for. Under such assumptions, the effective capacity C is therefore inversely related to the pressure increase rate P′.

The initial sieve bed pressure increase rate P′ may be measured by taking two samples P₁ and P₂ of sieve bed pressure at times t₁ and t₂ at the start of the pressurisation phase, and dividing the change in pressure by the elapsed time between t₁ and t₂:

$${P}^{′} = \frac{{P}_{2} - {P}_{1}}{{t}_{2} - {t}_{1}} \qquad ( 9 )$$

In some implementations, measurements of P′ may be compensated for variations in the mass flow rate Q between measurements of P′. A value for the mass flow rate Q may be obtained in a number of ways. One way is to use a mass flow sensor at the output of the compressor 200 to give a real-time-accurate measurement of Q. Another way is to use a function that calculates the mass flow rate Q from the current compressor characteristics, e.g. motor speed (measured by speed sensor 201), and current ambient conditions, such as one or more of temperature, barometric pressure, altitude, and humidity. Such a function may be developed during calibration of compressor 200 and embodied in, for example, a look-up table, such as a multi-dimensional look-up table, stored in memory 420 at the time of manufacture of the POC 100. Alternatively, such a function may be developed (for a reciprocating compressor 210) from the ideal gas law. Ambient conditions not available due to the absence of appropriate sensors may be set to typical values such as 20° C. for temperature, 70% for relative humidity, and sea level for altitude. A compensation factor may then be obtained by dividing the measured mass flow rate Q by a reference mass flow rate Q₀. The measurement of P′ may then be divided by the compensation factor.

In other implementations, the mass flow rate Q is set to the reference mass flow rate every time a measurement of P′ is made. However, in some implementations of a POC, during normal operation of the PSA cycle, the motor speed (which is directly related to the mass flow rate Q) is being repeatedly adjusted, such as by the fine pressure regulation scheme described above, to maintain the system pressure at a target pressure. Consequently, the mass flow rate Q may not be the same every time a measurement of P′ is made. Therefore, in such implementations, an effective capacity measurement mode may be invoked in which the motor speed is set to a predetermined value before a measurement of P′ is made. Assuming that there is a fixed relationship between compressor speed and output characteristics of the compressor such as flow rate, this is equivalent to setting the mass flow rate Q to a predetermined value.

In some implementations of a POC, such as a POC that uses the gas separation system 110, the initial sieve bed pressure increase rate P′ is not directly measurable, as the gas separation system does not have a pressure sensor within either canister 302 or 304. However, in implementations in which the pressure in the accumulator is measured by an accumulator pressure sensor such as the pressure sensor 107 in the gas separation system 110, the initial pressure increase rate in the accumulator may be measured using equation (9). This measurement is a reasonable proxy for the initial sieve bed pressure increase rate P′ under certain circumstances, principally that the check valve 142 or 144 is open and the supply valve 160 is closed. Therefore, the effective capacity measurement mode may be set up such that these conditions are met. The temperature in the accumulator is likely to be more stable than in the sieve bed, lessening the need for compensation of temperature variations.

In some implementations, the power parameter of the motor control signal 3030 to the motor driver 280 is representative of sieve bed pressure as described above. Therefore, in such implementations, as an alternative to using accumulator pressure, the power parameter of the motor control signal 3030 may be used as a proxy for sieve bed pressure in equation (9). In one such implementation, as described above, the power parameter is the duty cycle of the PWM waveform that acts as the motor control signal 3030.

Once an estimate of the sieve bed pressure increase rate P′ is available, the estimate may be converted to an estimate C of effective capacity by interpolation between values P₁′ and P₂′ for fresh and fully degraded sieve beds, respectively. A fully degraded sieve bed is a sieve bed whose output oxygen purity has fallen below an acceptable threshold, such as the threshold below which the oxygen enriched air is no longer regarded as medical oxygen, e.g. 80%.

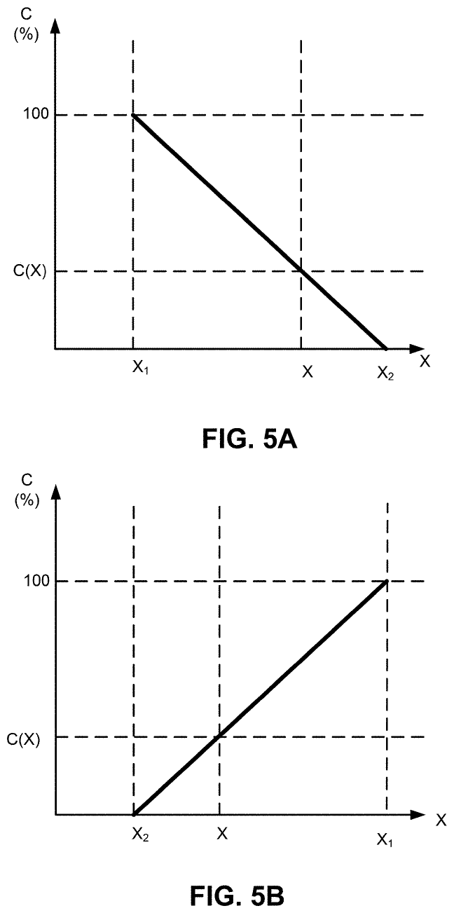

FIG. 5A contains a graph illustrating an inverse linear relationship between an operational parameter X of a sieve bed and the effective capacity C of the sieve bed. The value X₁ represents the value of the operational parameter obtained from a fresh sieve bed, at which the effective capacity is 1 (or 100%). The value X₂ represents the value of the operational parameter obtained from a fully degraded sieve bed, at which the effective capacity is 0%. The effective capacity C corresponding to an estimate X of the operational parameter may be obtained by linear interpolation between X₂ and X₁:

$$C = 100 \frac{{X}_{2} - X}{{X}_{2} - {X}_{1}} \qquad ( 10 )$$

FIG. 5B contains a graph illustrating a direct linear relationship between an operational parameter X of a sieve bed and the effective capacity C of the sieve bed. In FIG. 5B, as in FIG. 5A, the value X₁ represents the value of the operational parameter obtained from a fresh sieve bed, at which the effective capacity is 1 (or 100%), and the value X₂ represents the value of the operational parameter obtained from a fully degraded sieve bed, at which the effective capacity is 0%. If the operational parameter has this type of relationship with effective capacity, the effective capacity C corresponding to an estimate X of the operational parameter may be obtained by linear interpolation between X₁ and X₂:

$$C = 100 \frac{X - {X}_{2}}{{X}_{1} - {X}_{2}} \qquad ( 11 )$$

Linearity or inverse linearity is only one possible form of the relationship between the operational parameter X of a sieve bed and the effective capacity C of the sieve bed. Other forms of relationship between the operational parameter X of a sieve bed and the effective capacity C of the sieve bed, with consequent changes to the interpolation equations (10) and (11) are also contemplated.

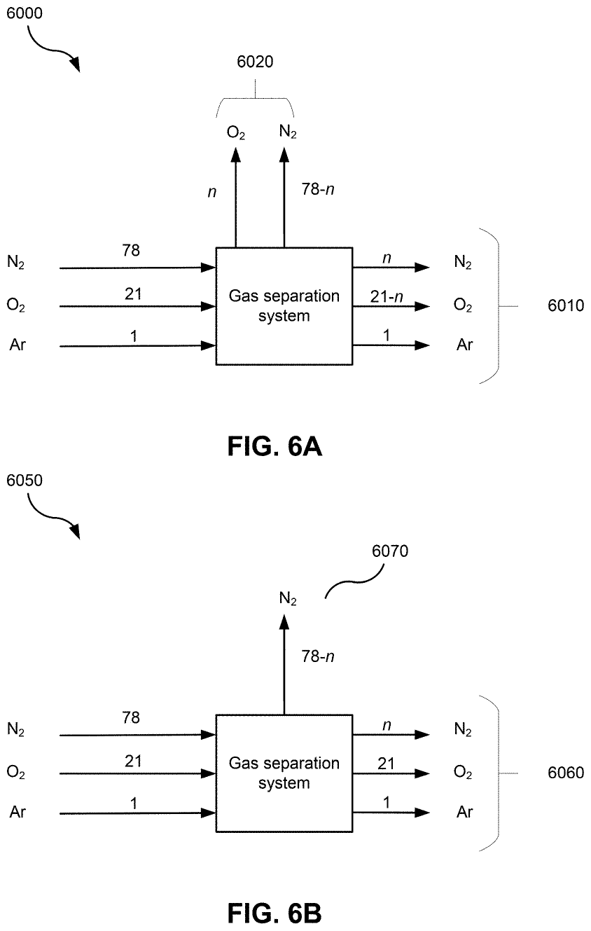

In some implementations, the values X₁ and X₂ may be obtained by measurement on fresh and fully degraded sieve beds respectively, provided the sieve beds are of the same type, and the measurement conditions the same, as the sieve bed for which the operational parameter X has been estimated. In some implementations, the value X₁ may be obtained by measurement on each fresh sieve bed as it is installed in the POC 100, provided the measurement conditions when the operational parameter X is estimated are the same as they were when the fresh sieve bed was installed. Alternatively, for some operational parameters, it is possible to calculate the value X₂ of the operational parameter from a fully degraded sieve bed from the value X₁ of the operational parameter obtained from a fresh sieve bed. In the case of the initial sieve bed pressure increase rate P′, such a calculation may be made based on models of gas flows in a gas separation system such as the models illustrated in FIGS. 6A and 6B.

FIG. 6A illustrates a model 6000 of gas flows in a gas separation system with a fresh sieve bed. The first assumption of the model 6000 is that of 100 moles of gas fed to the system, there are 78 moles of nitrogen, 21 moles of oxygen, and 1 mole of argon. Another assumption of the model 6000 is that the total amount of gas leaving the outlet 6010 for a fresh sieve bed is 22 moles, which includes all of the input argon. This amount also includes a small amount n of the input nitrogen, and therefore 21-n moles of the input oxygen, leaving 78-n moles of “separated” nitrogen and n moles of oxygen (totaling 78 moles) to exit via the exhaust outlet 6020 of the system. The amount n of output nitrogen varies with the degradation state of the sieve bed contained in the gas separation system. The output oxygen purity (the fraction of output oxygen mass flow over the sum of all output gas mass flows) is p, where p depends on n according to:

$$p = \frac{21 - n}{22} \qquad ( 12 )$$

Inverting equation (12), it may be shown that the amount n is equal to

n=21−22p (13)

For an assumed “fresh bed” output purity of 94%, n (the amount of outlet nitrogen and exhaust oxygen) evaluates to 0.32 moles.

FIG. 6B illustrates a model 6050 of gas flows in a gas separation system with a “fully degraded” sieve bed, in which the output purity is 80%. One assumption in the model 6050 is different from those in the model 6000, namely that all the oxygen coming in to the bed (21 moles) will exit the outlet 6060, since the available zeolite capacity is all taken by the nitrogen. This means that no oxygen exits the exhaust outlet 6070. Under the assumptions of the model 6050, and assuming a “fully degraded” output purity of 80%, n evaluates to 4.25 moles, so that the total output gas flow is 26.25 moles and the total exhaust flow is 73.75 moles.

The ratio of increase in output mass flow between fresh and fully degraded can be taken as an approximation to the ratio of increase of the mass flow of gas entering in the void, or the ratio of initial sieve bed pressure increase rate (since it is inversely related to zeolite adsorption). Therefore, the initial sieve bed pressure increase rate, is 26.25/22=1.19 times higher for a fully degraded sieve bed than for a fresh sieve bed based on the model 6000 in FIG. 6. In other words, when the operational parameter X is the initial sieve bed pressure increase rate, the value of X₂ for a fully degraded sieve bed may be estimated as 1.19 times the measured value X₁ for a fresh sieve bed.

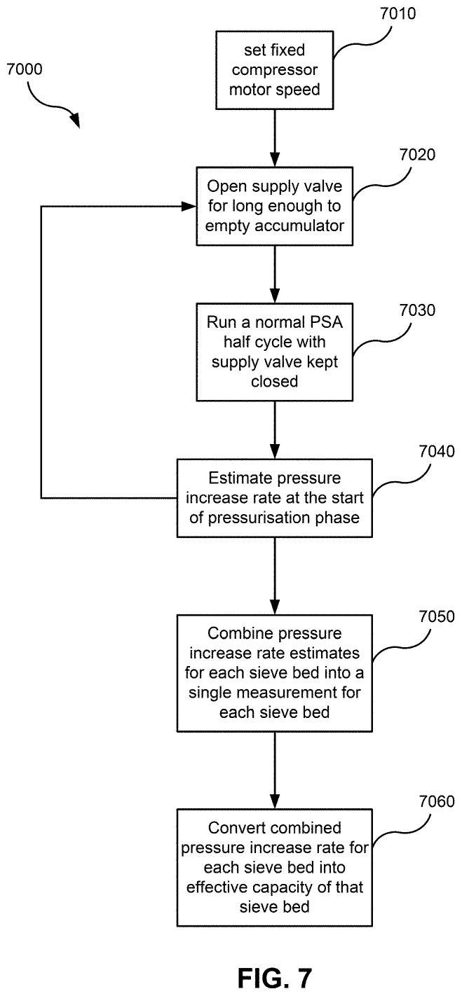

FIG. 7 contains a flow chart illustrating a method 7000 of estimating the effective capacity of the sieve beds of a POC such as the POC 100 in an effective capacity measurement mode according to one implementation of the present technology.

The method 7000 may start at step 7010, which fixes the motor speed by setting the speed set point 3010 to a predetermined value. The next step 7020 then opens the supply valve 160 for long enough to empty the accumulator of oxygen enriched air, which will be indicated by the accumulator pressure sensor 107 measuring the accumulator pressure to be ambient pressure (zero psi gauge). Step 7020 then closes the supply valve 160.

Step 7030 then runs a PSA half cycle as described above in relation to the state machine 4000, with the supply valve 160 kept closed. Because the accumulator pressure is at ambient, the check valve 142 or 144 opens immediately after the start of the pressurisation phase of the half cycle. At the start of the pressurisation phase, step 7040 estimates the pressure increase rate P_{A}′ in the sieve bed being pressurised (say sieve bed A in canister 302) using one of the proxy measurements described above (accumulator pressure, or power parameter of the motor control signal), e.g. using equation (9). The method 7000 then returns to step 7020 to empty the accumulator and make a measurement of the pressure increase rate P_{B}′ at the start of the pressurisation phase in the sieve bed B (canister 304). Steps 7020 to 7040 may be repeated as often as desired to yield multiple estimates of P_{A}′ and P_{B}′. If multiple estimates of either P_{A}′ or P_{B}′ have been obtained, those estimates may be combined into a single estimate P_{A}′ and P_{B}′ for each sieve bed, e.g. by averaging, at step 7050. The final step 7060 converts the combined estimates P_{A}′ and P_{B}′ into estimates of effective capacity C_{A} and C_{B} for the respective sieve beds A and B. Step 7060 uses equation (10), substituting P_{A}′ and P_{B}′ for since the initial sieve bed pressure increase rate is inversely related to effective capacity as described above.

In an alternative implementation, instead of using initial increase rate of sieve bed pressure, the rise time of sieve bed pressure during the pressurisation phase may be used. According to one model of the dynamic behaviour of the gas separation system (a resistor-capacitor or RC model), under the conditions of the effective capacity measurement mode, as the pressurisation phase continues, the sieve bed pressure increase rate decreases and the sieve bed pressure tends exponentially toward a maximum pressure Pₘₐₓ according to the following equation:

$$P = {P}_{max} ( 1 - {e}^{- \frac{f}{τ}} ) \qquad ( 14 )$$

where τ is the rise time of the sieve bed pressurisation. As the sieve bed degrades, more of the feed gas is available to increase the pressure in the sieve bed, and the rise time τ decreases. The rise time r is therefore directly related to the effective capacity of the sieve bed.

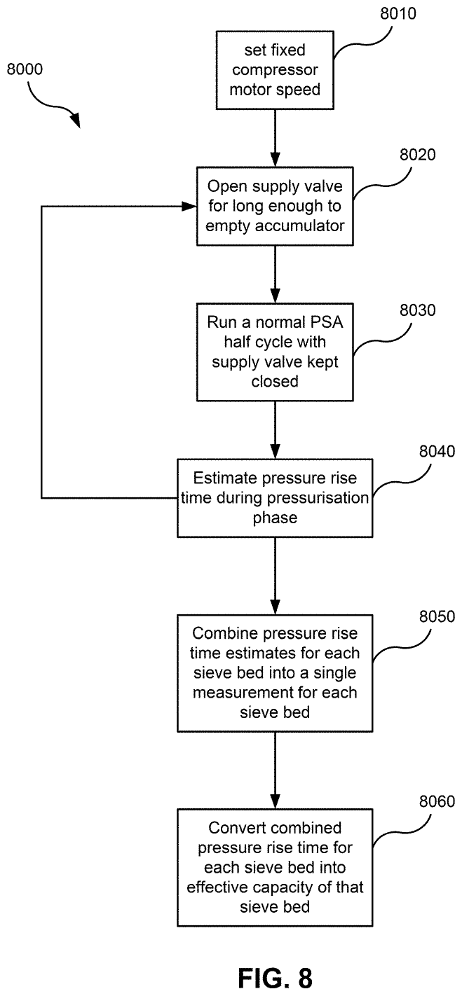

FIG. 8 contains a flow chart illustrating a method 8000 of estimating the effective capacity of the sieve beds of a POC such as the POC 100 in an effective capacity measurement mode according to one implementation of the present technology. The method 8000 is similar to the method 7000, with steps corresponding to the similarly numbered steps of the method 7000, with differences as described below.

Step 8040 estimates the pressure rise time τ in the sieve bed being pressurised using one of the proxy measurements described above (accumulator pressure, or power parameter of the motor control signal) from samples of the proxy measurement during the pressurisation phase. In some implementations, the rise time may be estimated from a set of samples by regression on the natural logarithm of the samples.

Step 8060 converts the combined rise time estimates τ_{A} and τ_{B} into estimates of effective capacity C_{A} and C_{B} for each sieve bed. Step 8060 uses equation (11), substituting τ_{A} and τ_{B} for X, since the sieve bed pressure rise time is directly related to effective capacity as described above. The values X₁ and X₂ may be obtained by measurement of rise time on fresh and fully degraded sieve beds respectively, provided the sieve beds are of the same type, and the measurement conditions the same, as the sieve beds for which rise times τ_{A} and τ_{B} were estimated. Alternatively, as for initial sieve bed pressure increase rate, it is possible to calculate the value X₂ of the rise time from a fully degraded sieve bed from the value X₁ of rise time obtained from a fresh sieve bed using the models 6000 and 6050 of FIGS. 6A and 6B. Because rise time is inversely proportional to initial sieve bed pressure increase rate under conditions of fixed motor speed, when the operational parameter X is the sieve bed pressure rise time, the value of X₂ may be estimated as 1/1.19 times the measured value X₁.

Other implementations of an effective capacity measurement mode make use of Equation (6). If a fully purged sieve bed were pressurised by a predetermined amount ΔP, then the total number N of moles of gas flowing into sieve bed, or out of it during the following depressurisation, would be made up of the n_{void} moles of equation (5) that entered the void, which is independent of the sieve bed degradation state, and the n_{ads} moles that were adsorbed by the adsorbent, which decreases as the sieve bed becomes more degraded and the effective capacity decreases. The total mass flow N may therefore be used as an operational parameter X that is directly related to the effective capacity of the sieve bed via Equation (11).

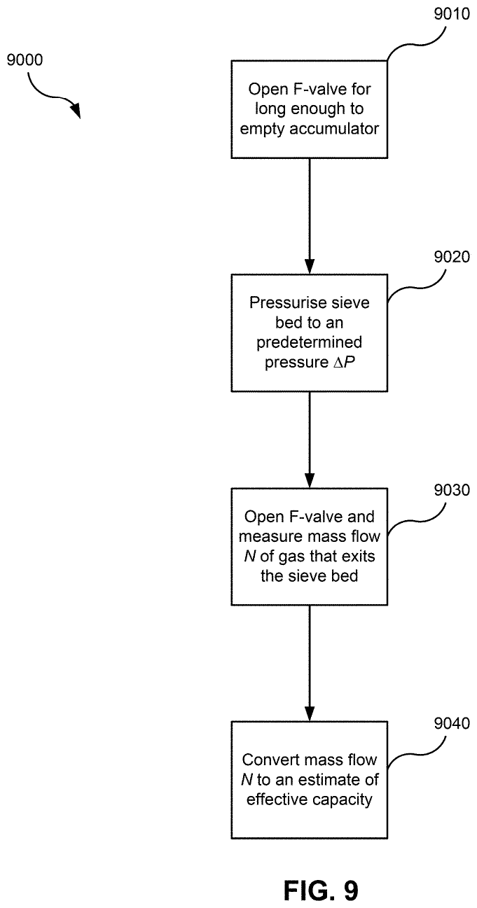

FIG. 9 contains a flow chart illustrating a method 9000 of estimating the effective capacity of the sieve beds of a POC such as the POC 100 using the total mass flow in an effective capacity measurement mode according to one implementation of the present technology.

The method 9000 is carried out at the end of a complete PSA half cycle, i.e. after an equalisation (2) phase, so that one of the sieve beds has been fully purged. Without loss of generality, the method will be described based on sieve bed B (canister 304) being the fully purged sieve bed.

Step 9010 opens the supply (F) valve 160 for long enough to empty the accumulator 106. Optionally, during this step the compressor motor speed may be reduced from its previous regulated level to speed up the emptying of the accumulator 106 and reduce the power consumed during the effective capacity measurement mode.

Step 9020 then pressurises sieve bed B to a predetermined pressure ΔP. This pressurisation may be monitored via the accumulator pressure sensor 107, and the compressor may be shut down when the sieve bed pressure has risen by ΔP.

Step 9030 then opens the supply valve 160 until the accumulator pressure falls to zero, and measures the total mass flow of gas that exits the pressurised sieve bed during this time. In some implementations, this measurement may be made by integrating the time profile of mass flow rate measured by an outlet mass flow rate sensor, if one is present. Alternatively, the supply valve 160 may be opened and closed repeatedly to release a series of boluses, and the mass flow of each released bolus may be estimated using an accumulator pressure/supply valve opening time modelling surface of bolus size as described above, where bolus size is number of moles. The total mass flow is the sum of the mass flows of all the boluses until the sieve bed and accumulator are depressurized. Optionally, the molar capacity of the accumulator 106 (if known) may be subtracted from the total mass flow to obtain the sieve bed mass flow N.

Finally, step 9040 converts the measured sieve bed mass flow N to an estimate of effective capacity C for the sieve bed under test using equation (11). The values X₁ and X₂ may be obtained by measurements of sieve bed mass flow on fresh and fully degraded sieve beds respectively, provided the sieve beds are of the same type, and the measurement conditions the same, as the sieve bed for which the mass flow was measured in step 9030.

In an alternative implementation of the method 9000, rather including a step 9030 to measure the mass flow of gas exiting the pressurised sieve bed, the mass flow of gas entering the sieve bed from the compressor during the pressurisation of step 9020 may be measured during step 9020. A mass flow sensor at the output of the compressor may be used for this purpose. In some implementations, this measurement may be made by integrating the time profile of mass flow rate measured by such a compressor output mass flow rate sensor.

In some implementations, effective capacity may be estimated from sieve bed pressure during normal operation of the POC, when fine pressure regulation is in operation to maintain the system pressure at a predetermined target pressure for a given flow rate setting via motor speed adjustments, rather than during an effective capacity measurement mode when the motor speed set point is fixed. One such implementation relies on the fact that fine pressure regulation is essentially choosing a mass flow rate Q that will maintain a constant sieve bed pressure increase rate P′ at the start of the pressurisation phase in order to reach the predetermined target pressure within the fixed timing of the pressurisation phase. The chosen mass flow rate Q is therefore inversely related to the void flow fraction x, according to equation (8), and directly related to the effective capacity C of the sieve beds. Since there is a direct monotonic (e.g. linear) relationship between compressor motor speed and the generated mass flow rate Q, the motor speed at a given flow rate setting under fine system pressure regulation is directly related to effective capacity. The motor speed may be measured directly using the signal generated by the motor speed sensor 201.

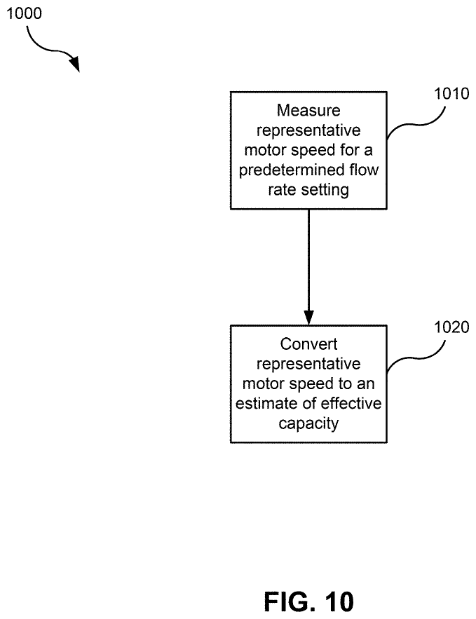

FIG. 10 contains a flow chart illustrating a method 1000 of estimating the effective capacity of the sieve beds of a POC such as the POC 100 using motor speed during normal operation of the fine pressure regulation mode according to one implementation of the present technology.

The method 1000 may start at step 1010, which takes a representative measurement of motor speed from the motor speed sensor 201 when the POC 100 is operating at a predetermined flow rate setting, e.g. setting 2. In one implementation, step 1010 averages a plurality of measurements of motor speed from the motor speed sensor 201 while the POC 100 is operating at the predetermined flow rate setting.

Step 1020 then converts the representative motor speed value from step 1010 to a measurement of collective effective capacity of the sieve beds of the POC 100. Step 1020 uses equation (11), substituting the representative motor speed for the operational parameter X, since the motor speed is directly related to effective capacity as described above. The values X₁ and X₂ may be obtained by measurement of motor speed on fresh and fully degraded sieve beds respectively, provided the sieve beds are of the same type, and the measurement conditions the same, as the sieve bed for which the representative motor speed was measured. Alternatively, as for initial sieve bed pressure increase rate, it is possible to calculate the value X₂ of the motor speed from a fully degraded sieve bed from the value X₁ of motor speed obtained from a fresh sieve bed using the models 6000 and 6050 of FIG. 6. Because motor speed under fine pressure regulation is analogous to initial sieve bed pressure increase rate under conditions of fixed motor speed, the value of X₂ of the motor speed from a fully degraded sieve bed may be estimated as 1.19 times the measured value X₁ from a fresh sieve bed.

In other implementations of effective capacity estimation during normal operation, exhaust flow may be used as an indicator of effective capacity. The rationale for such implementations is that a fresh sieve bed of high effective capacity will adsorb more gas, and will therefore exhaust more gas during depressurisation and purging, than a degraded sieve bed. The models 6000 and 6050 of FIGS. 6A and 6B may be used to show that for a fresh sieve bed (output purity p=94%), the amount of exhaust flow per 100 moles of input flow is 78 moles, while for a fully degraded sieve bed (output purity p=80%), the amount of exhaust flow per 100 moles of input flow is 73.75 moles, which is 5.8% lower than for a fresh sieve bed. These amounts of exhaust flow remain in the same proportion regardless of the input mass flow to the sieve bed. Consequently, the amount of exhaust flow is decoupled from the compressor flow rate, and depends only on the target pressure to which the sieve bed is increased during pressurisation, which is fixed for a given flow rate setting. As a result, the amount of exhaust flow (unlike the amount of outlet flow, or the sieve bed pressure increase rate or rise time) may be used to estimate effective capacity during normal operation of the PSA state machine.

Estimating effective capacity using exhaust flow may make use of a mass flow rate sensor in the exhaust outlet path (comprising the exhaust muffler 133 and the exhaust outlet 130) of the POC. A raw measurement of exhaust flow using such a sensor needs to be corrected for the amount of purge flow, since during the purge phase some of the oxygen enriched air from the adsorbing sieve bed is passed through the purged sieve bed to the exhaust outlet.

FIG. 11 contains a flow chart illustrating a method 1100 of estimating the effective capacity of the sieve beds of a POC such as the POC 100 during normal operation according to one implementation of the present technology.

The method 1100 may start at step 1110, which measures the total exhaust flow from a venting sieve bed over a PSA half cycle. In some implementations, this measurement may be made by integrating the time profile of mass flow rate measured by an exhaust mass flow rate sensor. Step 1120 then corrects this measurement of total exhaust flow by subtracting a value of purge mass flow. In some implementations, this purge mass flow value may be made by integrating the time profile of purge mass flow rate measured by a mass flow rate sensor in the purge flow path (comprising the G-valve 154 and the flow restrictors 153 and 155), if one is present. Alternatively, the purge mass flow value may be estimated based on an estimate of the pressure of the adsorbing sieve bed during its adsorption phase, along with a known pressure-flow characteristic of the purge flow path.

Steps 1110 and 1120 may be repeated multiple times over respective PSA half cycles for one or both of the sieve beds. If multiple estimates of corrected exhaust flow have been obtained for either sieve bed, those estimates may be combined into a single estimate for that sieve bed, e.g. by averaging, at step 1130.

The final step 1140 converts the (possibly) combined estimates of exhaust flow into estimates of effective capacity C_{A} and C_{B} for each sieve bed. Step 1140 uses equation (11), substituting the estimate of exhaust flow for X, since the total exhaust flow is directly related to effective capacity as described above. The values X₁ and X₂ may be obtained by measurement of total corrected exhaust flow on fresh and fully degraded sieve beds respectively, provided the sieve beds are of the same type, and the measurement conditions the same, as the sieve beds for which the total exhaust flows were estimated.

In other implementations of effective capacity estimation during normal operation, sieve bed pressure rise time may be used as an indicator of effective capacity. The variation in compressor motor speed resulting from the pressure regulation scheme affects the way the measured rise time varies with sieve bed degradation. The decrease in measured rise time as the effective capacity decreases is lessened because the motor speed needed to maintain the target pressure (and therefore the compressor output flow rate) is lessened also. However, the variation in motor speed may be estimated and the measured rise time compensated accordingly. One proxy for motor speed is the maximum pressure Pₘₐₓ towards which the sieve bed pressure tends under the RC model of the pressurisation phase (equation (14)). The value of Pₘₐₓ for a fully degraded sieve bed at a predetermined motor speed may be divided by the value of Pₘₐₓ for a fully degraded sieve bed with the pressure regulation scheme enabled. The measured rise time during normal operation may be divided by this ratio before applying equation (11) to estimate effective capacity.

Multiple estimates of effective capacity C(t₁), C(t₂), . . . C(t_{N}) at times t₁, t₂, . . . t_{N} may be converted to an estimate R of remaining usage time before full sieve bed degradation (zero effective capacity). In one implementation, a trend or time profile C(t) may be extracted from the estimates C(t₁), C(t₂), . . . C(t_{N}) of remaining capacity, and the time profile C(t) may be extrapolated to estimate the time to at which the remaining capacity C(t₀) will reach zero, assuming the continuance of a similar usage pattern that give rise to the estimates C(t₁), C(t₂), . . . C(t_{N}). The estimate R of remaining usage time may then be set to the difference between t₀ and the current time.

Compensation for Compressor Deterioration

Some of the apparatus and methods for estimating effective capacity described above rely on a fixed relationship between compressor speed and output characteristics of the compressor such as flow rate. However, over the lifetime of a compressor this relationship may change, for example due to leaks in the compressor seals. It would be advantageous to compensate for such changes in carrying out the methods described above, for this would reduce the effect of compressor deterioration as a source of inaccuracy in the estimation.

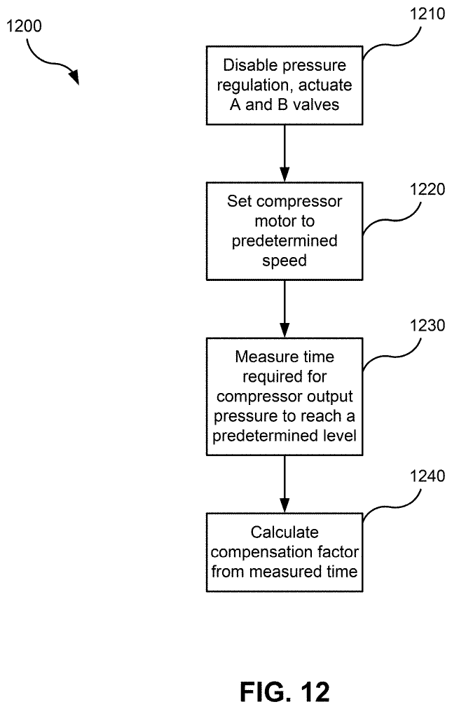

A compensation factor that may compensate for deterioration of the compressor may be estimated in a compressor characterisation mode. FIG. 12 contains a flow chart illustrating a method 1200 of characterising a compressor of a POC in a compressor characterisation mode according to one implementation of the present technology. The compressor characterisation mode may be entered, and the method 1200 initiated, when the POC is not in use, e.g. before shutdown.

The method 1200 may start at step 1210, which disables pressure regulation so that the compressor speed set point may be freely varied. Step 1210 also actuates the A- and B-valves 122 and 124 to disconnect the canisters 302 and 304 from the compressor. Step 1220 then sets the speed set point to a predetermined value. In some implementations, the predetermined value is an average of motor speed measurements taken at a predetermined flow rate setting, e.g. setting 2.

Step 1230 measures T, the time taken for the compressor output pressure to reach a predetermined level, for example 180 kPag. The predetermined level may be higher than the ordinary maximum pressure reached in the canisters during normal operation of the PSA cycle. In one implementation, the value of T may be measured by inserting a pressure relief valve in the flow path between the compressor and the A and B valves 122 and 124, with the pressure limit for the pressure relief valve pressure set to the predetermined level. The value of T is then the elapsed time from the execution of step 1220 until the pressure relief valve opens. In other implementations, T may be measured using the pressure measurement from a pressure sensor at the compressor output.

The value of T is inversely proportional to the output flow rate Q of the compressor. Step 1240 then computes a compensation factor from the measured value of T by dividing T by a benchmark measurement T₀ (representing the time taken for compressor output pressure to reach the predetermined level when the compressor was new). The measurement T₀ may be made, for example, during a calibration process on the compressor 210 of the POC 100, and stored in the memory 420. As the compressor deteriorates, the compensation factor, which starts out at 1, gradually increases above 1.

In an alternative implementation of a compressor characterisation mode, the compressor speed set point that allows the compressor output pressure to reach a predetermined level in a predetermined time (e.g. 1 second) may be measured. This is the compressor speed set point that causes the compressor to generate a predetermined output flow rate, and this speed set point tends to increase as the compressor deteriorates.

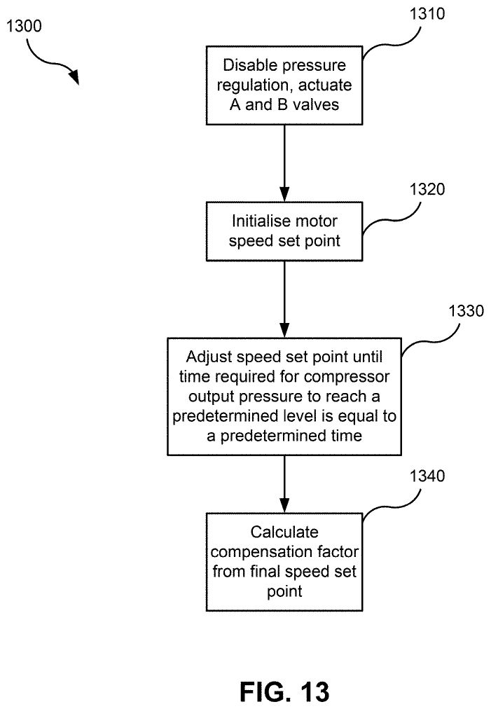

FIG. 13 contains a flow chart illustrating a method 1300 of characterising a compressor of a POC in a compressor characterisation mode according to one implementation of the present technology.

The method 1300 may start at step 1310, which disables pressure regulation so that the compressor speed set point may be freely varied. Step 1310 also actuates the A- and B-valves 122 and 124 to disconnect the canisters 302 and 304 from the compressor. Step 1320 then initialises the speed set point to a predetermined value. In some implementations, the predetermined value is an average of motor speed measurements taken at a predetermined flow rate setting, e.g. setting 2.

Step 1330 then adjusts the speed set point so that the time T taken for the compressor output pressure to reach a predetermined level, for example 180 kPag, equals a predetermined time T₁, for example one second. The value of T may be measured in similar fashion to step 1230 of the method 1200. Step 1330 may, in one implementation, use a PID controller to adjust the speed set point over repeated measurements of T until the measured value of T equals the predetermined time T₁.

Step 1340 then computes a compensation factor from the final speed set point R by dividing the final speed set point R by a benchmark measurement R₀ (representing the speed set point that allows the compressor output pressure to reach the predetermined level in the predetermined time when the compressor was new). The measurement R₀ may be made, for example, during a calibration process on the compressor 210 of the POC 100, and stored in the memory 420. As the compressor deteriorates, the compensation factor, which starts out at 1, gradually increases above 1.

The compensation factor calculated by the method 1200 or the method 1300 may be applied to the operation parameter X to compensate for any deterioration in the compressor before applying equation (10) or equation (11) to estimate the effective capacity of a sieve bed.

For example, the estimated initial sieve bed pressure increase rate values P_{A}′ and P_{B}′ may be multiplied by the compensation factor before applying equation (10) to estimate the effective capacity of a sieve bed at step 7060 of the method 7000. As another example, the representative motor speed may be divided by the compensation factor before applying equation (11) to estimate the effective capacity of a sieve bed at step 1020 of the method 1000.

Use of the Effective Capacity/Remaining Usage Time Estimate

The estimates of remaining sieve bed capacity and/or remaining usage time may be further utilised by the various entities in a connected oxygen therapy system such as the connected oxygen therapy system 450.

In one implementation, the effective capacity and/or remaining usage time estimate may be displayed on the control panel 600 of the POC 100. For example, the LEDs 655 may be used to indicate the current value of the effective capacity estimate (e.g. 100%, 75%, 50%, 25% as illustrated) rather than remaining battery power. This display may occur in response to activation of a separate button (not shown) on the control panel 600. Similarly, a numeric (e.g. 8-segment) display (not shown) could be used to display the current value of the effective capacity and/or remaining usage time estimate.

In another implementation, the “app” running on the portable computing device 480 could cause the value of the effective capacity and/or remaining usage time estimate to be displayed on a display of the portable computing device 480. This could occur on the instruction of the server 460 via a “push notification” to the app, or on the initiative of the app itself. Optionally, in some cases, the processor of the portable computing device may access data measured by the POC, such as by receiving such data from the POC, and compute the value of the effective capacity and/or remaining usage time estimate using any of the processing methodologies as previously described.

In a further implementation, the server 460 may be configured to host a portal system. The portal system may receive, from the portable computing device 480 or directly from the POC 100, data relating to the operation of the POC 100. For example, such operational data may include estimates of effective capacity or remaining usage time of sieve beds in a POC 100 or the measurements for computing such estimates at a server of the portal system. As described above, the personal computing device 464 may execute a client application such as a browser to allow a user of the personal computing device 464 (such as a representative of an HME) to access the operational data of the POC 100, and other POCs in the connected oxygen therapy system, via the portal system hosted by the server 460. In this fashion, such a portal system may be utilised by an HME to manage a population of users of POC devices, e.g. the POC device 100, in the connected oxygen therapy system.

The portal system may provide actionable insights into user or device condition for the population of POC devices and their users based on the operational data received by the portal system. Such insights may be based on rules that are applied to the operational data. In one implementation, the estimated remaining usage times of a fleet of POCs may be displayed to a representative of an HME on a display of a personal computing device 464 in a “window” of a client program interacting with the portal system. Further, a rule may be applied to each remaining usage time estimate. One example of such a rule is “If the remaining usage time for a POC is less than three weeks, highlight the POC in the display of remaining usage times”. Application of such a rule to the estimated remaining usage times results in the highlighting on the display of POCs with sieve beds approaching exhaustion. The highlighted POCs may then be noted by the HME for imminent sieve bed replacement. This is one example of the kind of rule-based fleet management made possible by the above-described methods of estimating sieve bed effective capacity operating within the connected oxygen therapy system.

Optionally, such as in case where the POC 100 determines an estimate of the effective capacity C of a sieve bed, the POC 100 may communicate a message, which may be based on the estimate, such as by a comparison with a threshold (e.g., if the estimate is at or below a threshold), to an external computing device of the system 450 such as to provide a notification message of a need for a sieve bed. Such a message may comprise a request for a new sieve bed such as for arranging a purchase or replacement order for a new sieve bed via an ordering or fulfillment system implemented with any of the devices of FIG. 7. Such a message may also be generated by any of the devices of the system 450 that receives either the estimate or the measurements and parameters necessary for determining the estimate. In such a case, the message may be further transmitted to other systems, such as a purchasing, ordering or fulfillment system or server(s) that may be configured to communicate with a device of the system 450 for arranging and/or completing such orders. Still further, in some versions, the POC may make a change in a control parameter of the POC based on the estimate or a comparison of the estimate and one or more thresholds. For example, one or more parameters for control of the PSA cycle of the POC may be adjusted based on the comparison. Such adjustments may include, for example, parameters for the various valve timings of the valves that control flow through the canisters for feed and purge cycles and/or compressor speed, etc. Such adjustments may be implemented for increasing remaining sieve bed usage life if a partially impaired bed is detected (e.g., less than 100%, 50% etc.) or resuming normal operating parameters for a detection of a renewed bed (e.g., greater than 50% or at or near 100%). Optionally, any of the devices of the system 450 may be configured to communicate command(s) to the POC for the POC to implement a change in a control parameter(s) of the POC, such as when such devices detect a need for such a change in the POC operation based on the estimate or a comparison of the estimate and one or more thresholds.

### Glossary

For the purposes of the present technology disclosure, in certain forms of the present technology, one or more of the following definitions may apply. In other forms of the present technology, alternative definitions may apply.

General

Air: In certain forms of the present technology, air may be taken to mean atmospheric air, consisting of 78% nitrogen (N₂), 21% oxygen (O₂), and 1% water vapour, carbon dioxide (CO₂), argon (Ar), and other trace gases.

Oxygen enriched air: Air with a concentration of oxygen greater than that of atmospheric air (21%), for example at least about 50% oxygen, at least about 60% oxygen, at least about 70% oxygen, at least about 80% oxygen, at least about 87% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Ambient: In certain forms of the present technology, the term ambient will be taken to mean (i) external of the treatment system or patient, and (ii) immediately surrounding the treatment system or patient.

Flow rate: The amount of gas passing a point per unit time. In some cases, a reference to flow rate will be a reference to a scalar quantity, namely a quantity having magnitude only. In other cases, a reference to flow rate will be a reference to a vector quantity, namely a quantity having both magnitude and direction.

Volumetric flow rate: The volume of gas passing a point per unit time, often measured in litres per minute. Standard volumetric flow rate is the volumetric flow rate under conditions of standard temperature and pressure.

Mass flow rate: The number of molecules of gas passing a point per unit time, often measured in moles per second. Mass flow rate and volumetric flow rate may be inter-converted if the temperature and pressure of the gas are known.

Flow therapy: Respiratory therapy comprising the delivery of a flow of air to an entrance to the airways at a controlled flow rate referred to as the treatment flow rate that is typically positive throughout the patient's breathing cycle.

Patient: A person, whether or not they are suffering from a respiratory disorder.

Pressure: Force per unit area. Pressure may be expressed in a range of units, including cmH₂O, g-f/cm², pounds per square inch (psi), and hectopascals. 1 cmH₂O is equal to 1 g-f/cm² and is approximately 0.98 hectopascal (1 hectopascal=100 Pa=100 N/m²=1 millibar ˜0.001 atm ˜0.15 psi). In this specification, unless otherwise stated, pressure values are given as gauge pressures (pressures relative to ambient pressure).

General Remarks

The term “coupled” as used herein means either a direct connection or an indirect connection (e.g., one or more intervening connections) between one or more objects or components. The phrase “connected” means a direct connection between objects or components such that the objects or components are connected directly to each other. As used herein the phrase “obtaining” a device means that the device is either purchased or constructed.

In the present disclosure, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative implementations of various aspects of the present technology may be apparent to those skilled in the art in view of this description. Accordingly, this description is to be construed as illustrative only and is for the purpose of teaching those skilled in the art the general manner of carrying out the technology. It is to be understood that the forms of the technology shown and described herein are to be taken as implementations. Elements and materials may be substituted for those illustrated and described herein, parts and processes may be reversed, and certain features of the technology may be utilized independently, all as would be apparent to one skilled in the art after having the benefit of this description of the technology. Changes may be made in the elements described herein without departing from the spirit and scope of the technology as described in the appended claims.

### REFERENCES

Ergun, S. (1952). Fluid flow through packed columns. Chem. Eng. Prog. 48.

Label list

| | |
|---|---|
| oxygen concentrator | 100 |
| inlet | 101 |
| inlet | 105 |
| accumulator | 106 |
| accumulator pressure sensor | 107 |
| inlet muffler | 108 |
| gas separation system | 110 |
| A-valve | 122 |
| B-valve | 124 |
| filter | 129 |
| concentrator exhaust outlet | 130 |
| muffler | 133 |
| spring baffle | 139 |
| check valve | 142 |
| flow restrictor | 143 |
| check valve | 144 |
| outlet system | 150 |
| E-valve | 152 |
| flow restrictors | 153 |
| G-valve | 154 |
| supply valve | 160 |
| oxygen sensor | 165 |
| outer housing | 170 |
| fan | 172 |
| outlet | 173 |
| outlet port | 174 |
| flow restrictor | 175 |
| power supply | 180 |
| particulate filter | 187 |
| connector | 190 |
| delivery conduit | 192 |
| pressure sensor | 194 |
| nasal cannula airway delivery device | 196 |
| mouthpiece | 198 |
| compression system | 200 |
| motor speed sensor | 201 |
| compressor | 210 |
| compressor outlet | 212 |
| motor | 220 |
| external rotatable armature | 230 |
| air transfer device | 240 |
| compressor outlet conduit | 250 |
| motor controller | 270 |
| motor driver circuit | 280 |
| load | 290 |
| canister system | 300 |
| purge canister | 302 |
| canister | 304 |
| air inlet | 306 |
| housing component | 310 |
| base | 315 |
| valve seat | 322 |
| openings | 323 |
| valve seat | 324 |
| outlet | 325 |
| exhaust gas | 327 |
| inlet conduit | 330 |
| valve seat | 332 |
| apertures | 337 |
| conduit | 342 |
| conduit | 344 |
| conduit | 346 |
| opening | 375 |
| controller | 400 |
| processors | 410 |
| memory | 420 |
| transceiver | 430 |
| system | 450 |
| server | 460 |
| analysis engine | 462 |
| personal computing device | 464 |
| database | 466 |
| network | 470 |
| portable computing device | 480 |
| program | 482 |
| user | 500 |
| housing component | 510 |
| conduit | 530 |
| conduit | 532 |
| opening | 542 |
| opening | 544 |
| valve seat | 552 |
| valve seat | 554 |
| control panel | 600 |
| input port | 605 |
| power button | 610 |
| flow rate setting button | 620 |
| flow rate setting button | 622 |
| button | 624 |
| button | 626 |
| active mode button | 630 |
| button | 635 |
| altitude button | 640 |
| battery check button | 650 |
| LEDs | 655 |
| method | 1000 |
| step | 1010 |
| step | 1020 |
| method | 1100 |
| step | 1110 |
| step | 1120 |
| step | 1130 |
| step | 1140 |
| method | 1200 |
| step | 1210 |
| step | 1220 |
| step | 1230 |
| step | 1240 |
| method | 1300 |
| step | 1310 |
| step | 1320 |
| step | 1330 |
| step | 1340 |
| PSA cycle | 2000 |
| valve actuation waveform | 2010 |
| waveforms | 2020 |
| valve actuation waveform | 2030 |
| pressure waveform | 2050 |
| pressure waveform | 2060 |
| motor control circuit | 3000 |
| speed set point | 3010 |
| speed signal | 3020 |
| motor control signal | 3030 |
| motor drive signal | 3040 |
| state machine | 4000 |
| models | 6000 |
| outlet | 6010 |
| exhaust outlet | 6020 |
| model | 6050 |
| outlet | 6060 |
| exhaust outlet | 6070 |
| method | 7000 |
| step | 7010 |
| step | 7020 |
| step | 7030 |
| step | 7040 |
| step | 7060 |
| method | 8000 |
| step | 8040 |
| step | 8060 |
| method | 9000 |
| step | 9010 |
| step | 9020 |
| step | 9030 |
| step | 9040 |

1. A method of estimating effective capacity of a sieve bed in an oxygen concentrator, the method comprising:
accessing a parameter of a measured pressure-time characteristic of the sieve bed for a phase of a pressure swing adsorption cycle of the oxygen concentrator at a predetermined speed of a motor of a compression system of the oxygen concentrator;
accessing one or more functions of the parameter of the measured pressure-time characteristic; and
estimating the effective capacity by applying the one or more functions to the parameter of the measured pressure-time characteristic.

2. The method of claim 1, wherein the one or more functions use a fresh value of the parameter, wherein the fresh value is a value of the parameter obtained from a fresh sieve bed of a same type as the sieve bed at the predetermined speed of the motor.

3. The method of claim 2, wherein the one or more functions use a fully degraded value of the parameter, wherein the fully degraded value is a value of the parameter obtained from a fully degraded sieve bed of a same type as the sieve bed at the predetermined speed of the motor.

4. The method of claim 3, wherein the one or more functions comprise an interpolation using the fresh value of the parameter and the fully degraded value of the parameter.

5. The method of claim 1, wherein the parameter is an initial rate of increase of the measured pressure-time characteristic.

6. The method of claim 1, wherein the parameter is a rise time of the pressure-time characteristic.

7. The method of claim 1, wherein the phase is a pressurisation phase of the pressure swing adsorption cycle.

8. The method of claim 1, further comprising measuring the pressure-time characteristic of the sieve bed for the phase of the pressure swing adsorption cycle of the oxygen concentrator.

9. The method of claim 8, wherein the measuring uses a pressure in an accumulator of the oxygen concentrator.

10. The method of claim 8, wherein the measuring uses a power parameter of a control signal of the motor.

11. The method of claim 1, further comprising:
repeating the accessing and the estimating to obtain a further estimate of effective capacity, and
estimating a remaining usage time of the sieve bed from the estimate and the further estimate of effective capacity.

12. The method of claim 1, further comprising displaying, on a display of the oxygen concentrator, an indicator of the estimated effective capacity.

13. The method of claim 1, further comprising generating a message based on the estimated effective capacity.

14. (canceled)

15. An oxygen concentrator comprising:
a sieve bed containing a gas separation adsorbent;
a compression system configured to feed a feed gas into the sieve bed;
a memory; and
a controller configured to:
access a parameter of a measured pressure-time characteristic of the sieve bed for a phase of a pressure swing adsorption cycle of the oxygen concentrator at a predetermined speed of a motor of a compression system of the oxygen concentrator;
access one or more functions of the parameter of the measured pressure-time characteristic; and
estimate effective capacity of the sieve bed by applying the one or more functions to the parameter of the measured pressure-time characteristic.

16. A connected oxygen therapy system comprising:
a portable oxygen concentrator comprising a sieve bed containing a gas separation adsorbent;
an external computing device in communication with the portable oxygen concentrator;
a memory; and
a processor configured by program instructions stored in the memory to estimate effective capacity of the sieve bed, the processor configured to:
access a parameter of a measured pressure-time characteristic of the sieve bed for a phase of a pressure swing adsorption cycle of the oxygen concentrator at a predetermined speed of a motor of a compression system of the oxygen concentrator;
access one or more functions of the parameter of the measured pressure-time characteristic; and
estimate effective capacity of the sieve bed by applying the one or more functions to the parameter of the measured pressure-time characteristic.

17. The connected oxygen therapy system of claim 16, wherein the processor and the memory are part of the portable oxygen concentrator.

18. The connected oxygen therapy system of claim 17, wherein the processor is further configured to transmit the effective capacity estimate to the external computing device.

19. The connected oxygen therapy system of claim 16, wherein the processor and the memory are part of the external computing device.

20. The connected oxygen therapy system of claim 16, further comprising a display.

21. The connected oxygen therapy system of claim 20, wherein the processor is further configured to display an indicator of the effective capacity that is estimated on the display.

22. The connected oxygen therapy system of claim 16, wherein the external computing device is a portable computing device.

23. The connected oxygen therapy system of claim 16, wherein the external computing device is a server.

24. The connected oxygen therapy system of claim 23, further comprising a personal computing device in communication with the server.

25. The connected oxygen therapy system of claim 24, wherein the personal computing device is configured to interact with a portal system hosted by the server.

26. The connected oxygen therapy system of claim 25, wherein the personal computing device is configured to:
receive the effective capacity estimate from the portal system; and
display the effective capacity estimate on a display of the personal computing device.

27. The connected oxygen therapy system of claim 23, further comprising a portable computing device in communication with the server.

28. The connected oxygen therapy system of claim 27, wherein the portable computing device is configured to:
receive the effective capacity estimate from the server; and
display the effective capacity estimate on a display of the portable computing device.

29. Apparatus comprising:
means for accessing a parameter of a measured pressure-time characteristic of a sieve bed for a phase of a pressure swing adsorption cycle of an oxygen concentrator at a predetermined speed of a motor of a compression system of the oxygen concentrator;
means for accessing one or more functions of the parameter of the measured pressure-time characteristic; and
means for estimating effective capacity of the sieve bed by applying the one or more functions to the parameter of the measured pressure-time characteristic.

30-76. (canceled)
